(19) United States
(12) Patent Application Publication
(10) Pub. No.: US 2010/0035932 A1
(43) Pub. Date: Feb. 11, 2010

(54) NOVEL FORMYL PEPTIDE RECEPTOR LIKE 1 AGONISTS THAT INDUCE MACROPHAGE TUMOR NECROSIS FACTOR ALPHA AND COMPUTATIONAL STRUCTURE-ACTIVITY RELATIONSHIP ANALYSIS OF THEREOF

(76) Inventors: Igor A. SCHEPETKIN, Bozeman, MT (US); Mark T. QUINN, Bozeman, MT (US); Liliya N. KIRPOTINA, Bozeman, MT (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/537,618

(22) Filed: Aug. 7, 2009

Related U.S. Application Data
(60) Provisional application No. 61/188,217, filed on Aug. 7, 2008.

(51) Int. Cl. A61K 31/443; C07D 405/12; C07D 307/78; A61K 31/357; A61P 35/00

(52) U.S. Cl. 514/336; 546/283.4; 549/435; 514/464

(57) ABSTRACT

The present invention provides compounds of structural formula (I), which are agonists of formyl peptide receptor (FPR), particularly formyl peptide receptor like 1 (FPRL1). The present invention also provides the therapeutic use of the compounds of formula (I).

### CROSS-REFERENCE TO RELATED APPLICATIONS

The present application claims priority to U.S. Provisional Patent Application No. 61/188,217, filed Aug. 7, 2008, which is hereby incorporated by reference in its entirety for all purposes.

### GOVERNMENT RIGHTS STATEMENT

This invention was made with United States Government support under Contract No. W9113M-04-1-0010 awarded by U.S. Army Space and Missile Defense Command (SMDC) and under Contract No. HHSN26620040009C awarded by National Institute of Health (NIH). The United States government has certain rights in this invention.

### FIELD OF THE INVENTION

The present invention generally relates to formal peptide receptor (FPR) agonists and therapeutic use thereof.

### BACKGROUND OF THE INVENTION

Tumor necrosis factor α (TNF-α) is a key cytokine in immune and inflammatory reactions and is important for both innate and adaptive immunity (Beutler, 1995). One of the most prominent characteristics of TNF-α is its ability to cause apoptosis of tumor-associated endothelial cells, resulting in tumor necrosis (Lejeune et al., 2006). However, despite its effectiveness against murine tumors (Old, 1985), clinical use of TNF-α has been limited due to its proinflammatory activity (Reed, 2006). On the other hand, stimulation of endogenous TNF-α production is still a reasonable approach in tumor biotherapy, and several compounds have been found to induce TNF-α and inhibit tumor blood flow in experimental tumors, with subsequent induction of necrosis (Baguley, 2001). Bacterial lipopolysaccharide (LPS) is a potent inducer of TNF-α; however, clinical trials with LPS have shown little success in cancer treatment (Jaeckle et al., 1990; Engelhardt et al., 1991). As an alternative to LPS, a number of small molecule cytokine inducers have been identified and characterized for their ability to stimulate TNF-α production. For example, both natural and synthetic agents with antimicrobial and antitumor properties, such as 5,6-dimethylxanthenone-4-acetic acid, acteoside, valine-proline boronic acid, ursolic acid, imidazoquinolines, and taxanes, have been shown to induce a broad range of cytokines in cell culture and/or in vivo (Burkhart et al., 1994; Wagner et al., 1997; Inoue et al., 1998; Joseph et al., 1999; Nemunaitis et al., 2006; Ikeda et al., 2007). Furthermore, induction of TNF-α by imidazoquinolines has been shown to be mediated through agonist activity toward Toll-like receptor (TLR)-7 and TLR-8 (Schön and Schön, 2007b). In contrast, not much is known regarding specific targets of most other reported small molecules that induce phagocyte TNF-α production.

The N-formyl peptide receptors (FPR) are a family of G-protein-coupled receptors (GPCR) involved in host defense and sensing cellular dysfunction (Migeotte et al., 2006; Rabiet et al., 2007). FPR are highly promiscuous receptors that can be activated by a wide range of structurally unrelated non-peptide and peptide agonists, including synthetic, or host-derived, and pathogen-derived agents (Migeotte et al., 2006; Rabiet et al., 2007). Three FPR subtypes are present in humans (FPR, FPRL1, FPRL2); whereas, eight FPR-related receptors have been identified in mice (Migeotte et al., 2006). Activation of FPR induces a variety of responses, which are dependent on the agonist, cell type, receptor subtype, and species involved. For example, N-formyl peptides, which are FPR and FPRL1 agonists, induce human phagocyte inflammatory responses, such as intracellular calcium mobilization, production of cytokines, generation of reactive oxygen species (ROS), and chemotaxis (Migeotte et al., 2006). In contrast, lipoxin A4 and related analogues, which are agonists of FPRL1, have been shown to promote resolution of inflammatory processes (Serhan, 2007). Although several, novel non peptide agonists of FPR/FPRL1 have been identified in recent year by high-throughput screening (Nanamori et al., 2004; Edwards et al., 2005; Burli et al., 2006; Zhou et al., 2007; Schepetkin et al., 2007), there are currently no known synthetic agonists of FPR/FPRL1 that activate phagocyte TNF-α production.

Structure-activity relationship (SAR) and quantitative SAR (QSAR) models have been instrumental in understanding of the molecular mechanism of action of receptor agonists and antagonists, directing their design, and in virtual screening. To date, non-computational SAR analysis has been performed for a series of taxoids; however, there are currently no reported computational SAR models for small-molecule inducers of TNF-α production.

While a variety of molecular parameters can be used in the computational methods for (Q)SAR analysis, some of these parameters are complex physicochemical or geometrical descriptors whose calculation is associated with difficulties due to molecular flexibility and inadequate sampling of conformational space. In contrast, topological indices (i.e., 2D descriptors) obtained from the structural formula of a compound are very attractive because of their simplicity. The present inventors have developed improved approach to SAR methodology based on atom pair descriptors in combination with classical physicochemical and geometrical descriptors and showed that this methodology can detect specific combinations of substructure patterns that confer high or low inhibitory activity against neutrophil elastase.

### SUMMARY OF THE INVENTION

In one embodiment, the present invention provides a compound of structural formula (I):

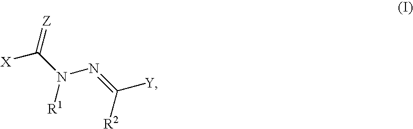

or a salt, solvate, ester and/or prodrug thereof, wherein X and Y are each independently aryl, substituted aryl, heteroaryl, or substituted heteroaryl; Z is oxygen or sulfur; R¹ is hydrogen or C1 to C6 alkyl; and R² is hydrogen, C1 to C6 alkyl, or R² and Y, taken together with the carbon atom to which they are bonded, form a substituted heterocyclic ring.

In another embodiment, the present invention provides an compound of structural formula (I) or a salt, solvate, ester and/or prodrug thereof, wherein X and Y are each independently aryl, substituted aryl, heteroaryl, or substituted heteroaryl; Z is oxygen or sulfur; R¹ is hydrogen or C1 to C6 alkyl; and R² is hydrogen, C1 to C6 alkyl, or R² and Y, taken together with the carbon atom to which they are bonded, form a substituted heterocyclic ring, and wherein the compound is active in inducing TNF-α in a human. An active compound can be differentiated from an inactive compound in that the active compound can induce macrophage TNF-α production, for example, showing an FI value of about 2 or higher.

In another embodiment, the present invention provides a pharmaceutical composition comprising a therapeutically effective amount of the compound of claim 1, or a salt, solvate, ester, and/or prodrug thereof; and a pharmaceutically acceptable carrier.

In another embodiment, the present invention provides a method of inducing apoptosis in a tumor-associated cell comprising contacting the tumor-associated cell with an effective amount of the compound of claim 1, or a salt, solvate, ester, and/or prodrug thereof.

In another embodiment, the present invention provides a method of treating a disease, condition, or symptom associated with TNF-α for a patient in need thereof comprising administering to the patient a therapeutically effective amount of the compound of claim 1, or a salt, solvate, ester, and/or prodrug thereof.

### BRIEF DESCRIPTION OF THE DRAWINGS

FIG. 1 depicts effect of selected arylcarboxylic acid hydrazides on TNF-α production by murine J774.A1 macrophages. Panel A: Macrophages (2×10⁵ cells/well) were cultured in the presence of the indicated concentrations of Compound 1 (), 2 (◯), 3 (▪), 4 (□), 5 (♦), AG-14 (Δ), or LPS (50 ng/ml) (for 24 hr, and TNF-α was measured in the cell supernatants by ELISA. Background due to control DMSO was negligible (not shown). Panel B: Macrophages (2×10⁵ cell/well) were cultured in the presence of the 12.5 μM Compound 1 (), 2 (◯), of DMSO (▪) for the indicated times, and TNF-α was measured in the cell supernatants by ELISA. The data in both panels are presented as the mean±SD of triplicate samples from one experiment, which is representative of three independent experiments.

FIG. 2 depicts effect of selected compounds on cell viability.

FIG. 3 depicts effect of actinomycin D and cycloheximide on Compound-induced TNF-α production by J774.A1 macrophages. J774.A1 macrophages were pretreated with actinomycin D (ActD; 1 μg/ml), cycloheximide (CHX; 5 μg/ml), or control DMSO for 30 min, followed by addition of 50 μM Compound 1 (open bars) or 2 (solid bars) for 12 hr. TNF-α was measured in the cell supernatants by ELISA. The data are presented as the mean±SD of triplicate samples from one experiment, which is representative of three independent experiments.

FIG. 4 depicts analysis of TNF-α production induced by Compounds 1 and 2 in murine peritoneal macrophages, human monocytic cells THP-1, human MonoMac6 macrophages, and human monocyte-derived macrophages. The indicated cells were incubated with Compound 1 (◯), 2 (), or LPS (50 ng/ml) (♦) for 24 hr, and TNF-α was measured in the cell supernatants by ELISA. Background due to control DMSO was negligible (not shown). The data are presented as the mean±SD of triplicate samples from one experiment, which is representative of three independent experiments.

FIG. 5 depicts effect of Compounds 1 and 2 on IL-6 production in murine J774.A1 macrophages. Macrophages (2×10⁵ cells/well) were cultured in the presence of Compound 1 (◯), 2 (), or LPS (50 ng/ml) (♦) for 24 hr, and TNF-α was measured in the cell supernatants by ELISA. Background due to control DMSO was negligible (not shown). The data are presented as the mean±SD of triplicate samples from one experiment, which is representative of three independent experiments.

FIG. 6 depicts neutrophil chemotactic activity of Compound 1 and Compound 2.

FIG. 7 depicts effect of Compounds 1 and 2 on Ca²⁺ mobilization in human neutrophils and J774.A1 macrophages. Panel A: Human neutrophils were labeled with FLIPR Calcium 3 dye, and changes in fluorescence were monitored after addition of 25 μM Compound 1 or 2, 5 nM fMLF, or control DMSO. Panel B: Macrophages loaded with Fura-2AM dye were treated with Compound 1 (◯) or 2 (), and changes in fluorescence were monitored (ex=340 nm and 380 nm, em=510 nm). The data are presented as the mean±SD of triplicate samples from one experiment, which is representative of three independent experiments.

FIG. 8 depicts analysis of receptor specificity for activation of Ca²⁺ mobilization by selected compounds. Panels A and B: RBL-FPR (Panel A) and RBL-FPRL1 (Panel B) cells were incubated with FLIPR Calcium 3 dye and the indicated concentrations of Compound 1 (), 2 (◯), or AG-14 (Δ), and [Ca²⁺]ᵢ flux was monitored for 5 min. Background due to control DMSO was negligible (not shown). The responses are presented as percent of response induced by EC₅₀ doses of fMLF (5 nM) or WKYMVm (10 pM) in RBL-FPR and RBL-FPRL1 cells, respectively. The data are representative of three independent experiments. Panel C: RBL-FPRL1 cells were labeled with FLIPR Calcium 3 dye, and changes in fluorescence were monitored after addition of Compound 1 (50 μM), 2 (50 μM), fMLF (5 nM), WKYMVm (50 pM), or control DMSO. The data are from one experiment, which is representative of three independent experiments.

FIG. 9 depicts characterization of TNF-α production induced by Compounds 1 and 2 in murine macrophages. Panel A: J774.A1 macrophages (2×10⁵ cells/well) were pretreated for 30 min with PTX (0.4 and 1 μg/ml), followed by the addition of Compound 1 (50 μM), 2 (50 μM), or LPS (50 ng/ml) for 12 hr, and TNF-α was measured in the cell supernatants by ELISA. Panel B: Macrophages (2×10⁵ cells/well) were cultured in the presence of fMLF (50 μM), WKYMVm (50 μM), Compound 1 (25 μM), or control DMSO for 24 hr, and TNF-α was measured in the cell supernatants by ELISA. Panel C: Macrophages (2×10⁵ cells/well) pretreated for 5 min with Boc 2 (40 μM) or WRW4 (10 μM), followed by the addition of Compound 1 (50 μM), 2 (50 μM), or LPS (50 ng/ml) for 24 hr. The data in all panels are presented as the mean±SD of triplicate samples from one experiment, which is representative of three independent experiments.

FIG. 10 depicts comparison of best-fit conformations of compounds 1 and 2 with the published threepoint pharmacophore model of FPR ligands. The conformations shown represent the best rootmean-square fit (ε) among all energy minima 6 kcal/mol above the global minimum. A1, A2, and H are the pharmacophore centers corresponding to the H-bond acceptors (A1 and A2) and hydrophobic center (pseudoatom H, green sphere), with permitted distances (Å) between points as follows: A1-A2 (3-6 Å), A1-H (5-7 Å), and A2-H (4-7 Å) (Edwards et al., 2005). For all structures, carbon atoms are sky-blue, nitrogen atoms are blue, oxygen atoms are red, hydrogen atoms are white, fluorine atoms are small yellow, and bromine are large yellow spheres.

FIG. 11 depicts chemical structures and activity of the most potent TNF-α inducers in J774.A1 macrophages.

FIG. 12 depicts effect of the most potent arylcarboxylic acid hydrazides on macrophage TNF-α production. J774.A1 macrophages (2×10⁵ cells/well) were cultured in the presence of the indicated concentrations of Compound 2 (□), Compound 51 (▪), or 50 ng/ml LPS () for 24 hr, and TNF-α was measured in the cell supernatants by ELISA. The data are presented as the mean±SD of triplicate samples from one experiment, which is representative of three independent experiments.

FIG. 13 depicts examples of atom pair descriptors in selected active arylcarboxylic acid hydrazides. Atom pairs are depicted in red and indicated below the structure. Compound numbers correspond to those shown in Table 5.

FIG. 14 depicts numbers of unique atom pairs in the set of arylcarboxylic acid hydrazides. The numbers are shown for each of the indicated bond distances initially generated for the 86 hydrazides (Panel A). Atom pairs subsequently included in the best LDA model are shown in Panel B.

FIG. 14 depicts examples of descriptors with one-dimensional (A) and two-dimensional (B) separation. Active and non-active compounds are represented by open and close circles, respectively.

FIG. 16 depicts binary classification tree reflecting the simplified SAR rules for predicting macrophage TNF-α inducing activity of arylcarboxylic acid hydrazide derivatives.

### DETAILED DESCRIPTION OF THE INVENTION

### Definitions

As used herein, the phrase “tumor necrosis factors” refers to a group of cytokines family that can cause apoptosis.

As used herein, the term ‘cytokines” refers to a category of signaling molecules that are used in cellular communication. They are proteins, peptides, or glycoproteins. The term cytokine encompasses a large and diverse family of regulators that are produced widely throughout the body by cells of diverse embryological origin. The action of cytokines may be autocrine, paracrine, and endocrine. Cytokines are critical to the development and functioning of both the innate and adaptive immune response, although not limited to just the immune system. Cytokines are also involved in several developmental processes during embryogenesis.

As used herein, the term “apoptosis” refers to the process of programmed cell death, which involves a series of biochemical events leading to characteristic cell morphology changes including blebbing, changes to the cell membrane such as loss of membrane asymmetry and attachment, cell shrinkage, nuclear fragmentation, chromatin condensation, and chromosomal DNA fragmentation, and to cell death.

As used herein, the term “neoplasm” (i.e., neoplastic disease) or “tumor” refers to a neoplastic mass resulting from abnormal cell growth, which can be benign or malignant. Benign tumors generally remain localized. Malignant tumors generally have the potential to invade and destroy neighboring body tissue and spread to distant sites and cause death (for review, see Robins and Angell, 1976, Basic Pathology, 2d Ed., W.B. Saunders Co., Philadelphia, pp. 68-122). A tumor is said to have metastatized when it has spread from one organ or tissue to another. Examples of solid tumors that can be treated according to the invention include sarcomas and carcinomas such as, but not limited to: fibrosarcoma, myxosarcoma, chondrosarcoma, osteogenic sarcoma, angiosarcoma, endotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, renal cell carcinoma, hepatoma, bile duct carcinoma, cervical cancer, testicular tumor, lung carcinoma, bladder carcinoma, epithelial carcinoma, melanoma, and retinoblastoma.

As used herein, the phrase “anti-tumor activity” is defined as any reduction in tumor mass or tumor burden after administration of the compounds/agents of the present invention or formulations pursuant to the present invention

As used herein, the phrase “systemic administrations” refers to parenteral, topical, oral, spray inhalation, rectal, nasal and, buccal administration.

As used herein, the phrase “parenteral administrations” refers to subcutaneous, intravenous, intramuscular, intra-articular, intra-synovial, intrasternal, intrathecal, intrahepatic, intralesional and intracranial administration.

As used herein, the phrase “therapeutically effective amount” is used herein to mean an amount sufficient to reduce by at least about 15%, 20%, 25%, 30%, 35%, 40%, 45%, 50%, 55%, 60%, 65%, 70%, 75%, 80%, 85%, 90%, 95%, 98%, 99%, 99.5%, or prevent growth and/or metastasis of a tumor and a clinically significant deficit in the activity, function and response of the host. Alternatively, a therapeutically effective amount is sufficient to cause an improvement in a clinically significant condition in the host e.g., a reduction in the tumor burden. The “therapeutically effective amount” will vary depending on the compound, the disease and its severity and the age, weight, etc., of the patient to be treated.

As used herein, the phrase “therapeutically effective” or “effective amount” applied to dose or amount refers to that quantity of a compound or pharmaceutical composition that is sufficient to result in a desired activity upon administration to a mammal or contacting with a cell in need thereof. More specifically, the term “therapeutically effective” refers to that quantity of a compound or pharmaceutical composition that is sufficient to reduce or eliminate a tumor in a mammal. Note that when a combination of active ingredients is administered the effective amount of the combination may or may not include amounts of each ingredient that would have been effective if administered individually.

The term “alkyl,” by itself or as part of another substituent, refers to a branched, straight-chain, or cyclic monovalent hydrocarbon radical derived by the removal of one hydrogen atom from a single carbon atom of a parent alkane. In some embodiments of the invention, the alkyl groups are “C1 to C6 alkyl”, i.e., alkyl group having 1 to 6 carbon atoms, such as methyl, ethyl, propyl, isopropyl, n-butyl, iso-butyl, sec-butyl, tert-butyl, amyl, tert-amyl, hexyl and the like. Many embodiments of the invention comprise “C1 to C4 alkyl” groups (alternatively termed “lower alkyl” groups) that include, but are not limited to, methyl, ethyl, propyl, iso-propyl n-butyl, iso-butyl, sec-butyl, and t-butyl groups. Some of the preferred alkyl groups of the invention have three or more carbon atoms preferably 3 to 16 carbon atoms, 4 to 14 carbon atoms, or 6 to 12 carbon atoms. Typical alkyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

The term “alkoxy” refers to an —OR radical or group, wherein R is an alkyl radical. In some embodiments the alkoxy groups can be C1 to C8, and in other embodiments can be C1 to C4 alkoxy groups wherein R is a lower alkyl, such as a methoxy, ethoxy, n-propoxy, isopropoxy, n-butoxy, t-butoxy and like alkoxy groups. The alkoxy group may be optionally further substituted meaning that the R group is a substituted alkyl group or residue. Examples of substituted alkoxy groups include trifluoromethoxy, hydroxymethyl, hydroxyethyl, hydroxypropyl, and alkoxyalkyl groups such as methoxymethyl, methoxyethyl, polyoxoethylene, polyoxopropylene, and similar groups.

The term “amino” refers to —NH₂, while the term “alkylamino” refers to —NHR or —NR₂, wherein each R is independently an alkyl radical. In some embodiments the alkylamine groups can be C1 to C8, and in other embodiments can be C1 to C4 alkylamine groups wherein R is a lower alkyl, such as a methylamine, ethylamine, propylamine, N,N-methylethylamine, N,N-dimethylamine, N,N-diethylamine, N,N-diisopropylamine and like alkylamine groups.

The term “heterocyclyl” or “heterocyclic ring” denotes optionally substituted 3 to 8-membered rings having one or more carbon atoms connected in a ring that also comprise 1 to 5 ring heteroatoms, such as oxygen, sulfur and/or nitrogen inserted into the ring. These heterocyclic rings can be saturated, unsaturated or partially unsaturated, but are preferably saturated. Preferred unsaturated heterocyclic rings include furanyl, thiofuranyl, pyrrolyl, pyridyl, pyrimidyl, pyrazinyl, benzoxazole, benzthiazole, quinolinlyl, and like heteroaromatic rings. Preferred saturated heterocyclic rings include piperidyl, aziridinyl, piperidinyl, piperazinyl, tetrahydrofurano, pyrrolyl, and tetrahydrothiophenyl rings.

An “aryl” group refers to a monocyclic, linked bicyclic or fused bicyclic radical or group comprising at least one six membered aromatic “benzene” ring. Aryl groups preferably comprise between 6 and 12 ring carbon atoms, and are exemplified by phenyl, biphenyl, naphthyl, indanyl, and tetrahydronapthyl groups. Aryl groups can be optionally substituted with various organic and/or inorganic substituent groups, wherein the substituted aryl group in combination with all its substituents comprise between 6 and 18, or preferably 6 and 16 total carbon atoms. Examples of the optional substituent groups include 1, 2, 3, or 4 substituent groups independently selected from hydroxy, fluoro, chloro, NH₂, NHCH₃, N(CH₃)₂, CO₂CH₃, nitro, cyano, methyl, ethyl, isopropyl, vinyl, trifluoromethyl, methoxy, ethoxy, isopropoxy, and trifluoromethoxy groups. In some embodiments, the optional substituent is a ring fused into the aromatic “benzene” ring, such as dioxole and dioxine.

The term “heteroaryl” means a heterocyclic aryl derivative which preferably contains a five-membered or six-membered conjugated and aromatic ring system having from 1 to 4 heteroatoms independently selected from oxygen, sulfur and/or nitrogen, inserted into the unsaturated and conjugated heterocyclic ring. Heteroaryl groups include monocyclic heteroaromatic, linked bicyclic heteroaromatic, or fused bicyclic heteroaromatic moieties. Examples of heteroaryls include pyridinyl, pyrimidinyl, and pyrazinyl, pyridazinyl, pyrrolyl, furanyl, thiofuranyl, oxazoloyl, isoxazolyl, phthalimido, thiazolyl, quinolinyl, isoquinolinyl, indolyl, or a furan or thiofuran directly bonded to a phenyl, pyridyl, or pyrrolyl ring and like unsaturated and conjugated heteroaromatic rings. Any monocyclic, linked bicyclic, or fused bicyclic heteroaryl ring system which has the characteristics of aromaticity in terms of electron distribution throughout the ring system is included in this definition. Typically, the heteroaromatic ring systems contain 3-12 ring carbon atoms and 1 to 5 ring heteroatoms independently selected from oxygen, nitrogen, and sulfur atoms.

The terms “arylalkyl” and “heteroarylalkyl” refer to aromatic and heteroaromatic systems, respectively, which are coupled to another residue through a carbon chain, including substituted or unsubstituted, saturated or unsaturated, carbon chains, typically of one to six carbon atoms. These carbon chains may optionally include a carbonyl group, thus making them able to provide substituents as an acyl moiety. Preferably, arylalkyl or heteroarylalkyl is an alkyl group substituted at any position by an aryl group, substituted aryl, heteroaryl or substituted heteroaryl. Examples of arylalkyl and heteroarylalkyl include benzyl, 2-phenylethyl, 3-phenyl-propyl, 4-phenyl-n-butyl, 3-phenyl-n-amyl, 3-phenyl-2-butyl, 2-pyridinylmethyl, 2-(2-pyridinyl)ethyl, and the like.

The term “halo” or “halogen” refers to fluoro, chloro, bromo or iodo atoms or ions. Preferred halogens are chloro and fluoro.

As used herein, the term “substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, aryl, arylalkyl, heteroaryl, heteroarylalkyl, and heterocyclic; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered heterocyclic ring which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and heterocyclyl. In some embodiments, the substituents of substituted aryl, substituted heteroaryl, or substituted heterocyclyl, taken together with the carbon atoms to which they are bonded, form a cycloalkyl or heterocyclic ring.

“Pharmaceutically acceptable” refers to being suitable for use in contact with the tissues of humans and animals without undue toxicity, irritation, allergic response, and the like, commensurate with a reasonable benefit/risk ratio, and effective for their intended use within the scope of sound medical judgment.

The term “carrier” refers to a diluent, adjuvant, excipient or vehicle with which a compound is administered.

One or more of the compounds of the invention, may be present as a salt. The term “salt” encompasses those salts that form with the carboxylate anions and amine nitrogens and include salts formed with the organic and inorganic anions and cations discussed below. Furthermore, the term includes salts that form by standard acid-base reactions with basic groups (such as nitrogen containing heterocycles or amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and like acids.

By “solvate”, it is meant a complex formed by solvation (the combination of solvent molecules with molecules or ions of the present compounds), or an aggregate that consists of a solute ion or molecule of the present compounds with one or more solvent molecules. When water is the solvent, the corresponding solvate is “hydrate”.

The term “ester” refers to any ester of the present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, heteroaryl, arylalkyl, heteroarylalkyl, heterocyclyl, heterocyclylalkyl, and substituted derivatives thereof. The term “ester thereof” includes but is not limited to pharmaceutically acceptable esters thereof.

The term “prodrug” refers to a precursor of the active agent wherein the precursor itself may or may not be pharmaceutically active but, upon administration, will be converted, either metabolically or otherwise, into the active agent or drug of interest. For example, prodrug includes an ester or an ether form of an active agent.

The term “patient” includes mammal, preferably human.

The term “optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyls where there is substitution.

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The term “FI” refers to the fold-increase of macrophage TNF-α production induced by 50 μM of a compound of the present invention compared to vehicle (DMSO) control.

### Cellular Immunity and Cytokines

One strategy for the treatment of cancer involves enhancing or activating a cellular immune response. Successful induction of a cellular immune response directed toward autologous tumors offers several advantages over conventional chemotherapy: 1) immune recognition is highly specific, being directed exclusively toward tumors; 2) growth at metastatic sites can be suppressed through immune surveillance; 3) the diversity of immune response and recognition can compensate for different resistance mechanisms employed by tumor cells; 4) clonal expansion of cytotoxic T cells can occur more rapidly than the expanding tumor, resulting in antitumor mechanisms which ultimately overwhelm the tumor; and 5) a memory response can suppress disease recurrence in its earliest stages, prior to physical detection. Clinical studies of responding patients have borne out results from animal models demonstrating that successful immunotherapy involves the activation of CD8+ T cells (class I response), although evidence exists for participation of CD4+ T cells, macrophages, and NK cells. See, e.g., Chapoval et al., 1998, Gollub et al., 1998, Kikuchi et al., 1999, Pan et al., 1995, Saffran et al., 1998 and Zimmermann et al., 1999.

### Tumor Necrosis Factor (TNF) Family of Cytokines

The best characterized member of the TNF family is TNF-α. TNF-α is known to exert pleiotropic effects on the immune system. TNF-α is a cytokine which can exert potent cytotoxic effects directly on tumor cells. TNF-α is generally thought to exert its anti-tumor effects via other mechanisms such as stimulation of proliferation and differentiation, and prevention of apoptosis in monocytes (see, e.g., Mangan et al., 1991, J. Immunol. 146:1541-1546; and Ostensen et al., 1987, J. Immunol. 138:4185-4191), promotion of tissue factor-like procoagulant activity and suppression of endothelial cell surface anticoagulant activity, ultimately leading to clot formation within the tumor (reviewed in Beutler and Cerami, 1989, Ann. Rev. Immunol. 7:625-655; and Vassalli, P., 1992, Ann. Rev. Immunol. 10:411-452). However, as a result of these properties, systemic administration of TNF-α results in lethal consequences in the host due to disseminated intravascular coagulation. Both TNF-α and IL-2 aid in lymphocyte homing. In the presence of both TNF-α and IL-2, the cytolytic activity of NK and LAK cells is increased, even when directed against TNF-insensitive cell lines (see, e.g, Ostensen et al., 1987, J. Immunol. 138:4185-4191).

### Formyl Peptide Receptor Agonists

The present invention herein provides novel and inventive agonists of formyl peptide receptor (FPR), particularly formyl peptide receptor like 1 (FPRL1). In one embodiments, the FPR agonists of the present invention are non-peptide and non-naturally occurring (i.e., synthetic) compounds.

In one embodiment, the compounds of the present invention has a structural formula (I):

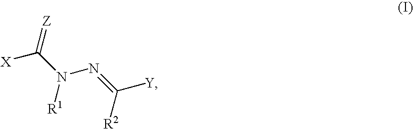

or a salt, solvate, ester and/or prodrug thereof, wherein X and Y are each independently aryl, substituted aryl, heteroaryl, or substituted heteroaryl; Z is oxygen or sulfur; R¹ is hydrogen or C1 to C6 alkyl; and R² is hydrogen, C1 to C6 alkyl, or R² and Y, taken together with the carbon atom to which they are bonded, form a substituted heterocyclic ring.

In one embodiment of formula (I), Z is oxygen.

In another embodiment of formula (I), R¹ is hydrogen.

In another embodiment of formula (I), R² is hydrogen.

In another embodiment of formula (I), Z is oxygen; R¹ is hydrogen; and R² is hydrogen.

In another embodiment of formula (I), Z is oxygen; R¹ is hydrogen; and R² and Y, taken together with the carbon atom to which they are bonded, form a substituted heterocyclic ring.

In another embodiment of formula (I), aryl is phenyl or naphthyl.

In another embodiment of formula (I), substituted aryl comprises one or more substituents selected from the group consisting of alkyl, substituted alkyl, aryl, substituted aryl, heteroaryl, substituted heteroaryl, hydroxy, halo, nitro, cyano, amino, alkylamino, —OR³, and a combination thereof; and optionally two of the substituents, taken together with the carbon atoms to which they are bonded, form a heterocyclic ring; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—. In one specific embodiment, two of the substituents, taken together with the carbon atoms to which they are bonded, form a dioxole or dioxine.

In another embodiment of formula (I), heteroaryl comprises a 5- or 6-membered aromatic ring having 1 to 3 heteroatoms selected from the group consisting of nitrogen, oxygen, sulfur, and a combination thereof. In one specific embodiment, heteroaryl is selected from the group consisting of furan, pyrrole, thiophene, imidazole, oxazole, thiazole, pyridine, pyrazine, and pyrimidine.

In another embodiment of formula (I), substituted heteroaryl comprises one or more substituents selected from the group consisting of alkyl, substituted alkyl, aryl, substituted aryl, heteroaryl, substituted heteroaryl, hydroxy, halo, nitro, cyano, amino, alkylamino, —OR³, and a combination thereof; and optionally two of the substituents, taken together with the carbon atoms to which they are bonded, form a heterocyclic ring; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—.

In another embodiment of formula (I), wherein X is aryl, substituted aryl, heteroaryl, or substituted heteroaryl; wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof; Y is heteroaryl or substituted heteroaryl; wherein substituted heteroaryl comprises one or more substituents selected from the group consisting of alkyl, substituted alkyl, aryl, substituted aryl, heteroaryl, substituted heteroaryl, alkoxy, hydroxy, halo, nitro, cyano, amino, alkylamino, and a combination thereof; Z is oxygen; and R¹ and R² are both hydrogen.

In another embodiment of formula (I), wherein X is substituted aryl which comprises two or more substituents, wherein two of the substituents, taken together with the carbon atoms to which they are bonded, form a heterocyclic ring; Y is heteroaryl or substituted heteroaryl; wherein substituted heteroaryl comprises one or more substituents selected from the group consisting of alkyl, substituted alkyl, aryl, substituted aryl, heteroaryl, substituted heteroaryl, alkoxy, hydroxy, halo, nitro, cyano, amino, alkylamino, and a combination thereof; Z is oxygen; and R¹ and R² are both hydrogen.

In another embodiment of formula (I), wherein the compound has structural formula (II):

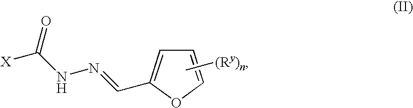

wherein X is aryl, substituted aryl, heteroaryl, or substituted heteroaryl; each R^{y} is independently alkyl, substituted alkyl, aryl, substituted aryl, heteroaryl, substituted heteroaryl, alkoxy, hydroxy, halo, nitro, cyano, amino, or alkylamino; and n is 0, 1, 2, or 3.

In one embodiment of formula (II), wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof; and optionally two of the substituents, taken together with the carbon atoms to which they are bonded, form a heterocyclic ring;

In another embodiment of formula (I), wherein X is aryl, substituted aryl, heteroaryl, or substituted heteroaryl; wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof; Y is aryl or substituted aryl; wherein substituted aryl comprises one or more substituents selected from the group consisting of alkyl, substituted alkyl, hydroxy, halo, nitro, cyano, amino, alkylamino, —OR³, and a combination thereof; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—; Z is oxygen; and R¹ and R² are both hydrogen.

In another embodiment of formula (I), wherein X is substituted aryl which comprises two or more substituents, wherein two of the substituents, taken together with the carbon atoms to which they are bonded, form a heterocyclic ring; Y is aryl or substituted aryl; wherein substituted aryl comprises one or more substituents selected from the group consisting of alkyl, substituted alkyl, hydroxy, halo, nitro, cyano, amino, alkylamino, —OR³, and a combination thereof; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—; Z is oxygen; and R¹ and R² are both hydrogen.

In another embodiment of formula (I), wherein the compound has structural formula (III):

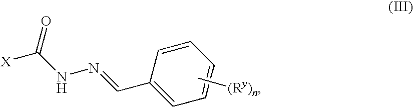

wherein X is aryl, substituted aryl, heteroaryl, or substituted heteroaryl; each R^{y} is independently alkyl, substituted alkyl, hydroxy, halo, nitro, cyano, amino, alkylamino, or —OR³; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—; and n is 0, 1, 2, or 3.

In another embodiment of formula (I), wherein X is substituted aryl, which comprises two or more substituents wherein two of the substituents, taken together with the carbon atoms to which they are bonded, form a heterocyclic ring; Z is oxygen; R¹ is hydrogen; and R² and Y, taken together with the carbon atom to which they are bonded, form a substituted heterocyclic ring.

In a specific embodiment, the compound of the present invention is selected from the group consisting of the compounds as described in the Examples hereinbelow.

The compounds of the present invention also include their tautomers and stereoisomers, such as cis- and trans-isomer, E- or Z-isomers, diastereomers, enantiomers, and mixtures thereof. These compounds can be prepared via synthetic methods commonly known to one skilled in the art.

### Pharmaceutical Formulations and Administration

The compounds of the present invention can be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients, such as the compound of the present invention, to be administered alone it may be preferable to present them as pharmaceutical formulations. The formulations of the invention, both for veterinary and for human use, comprise at least one active ingredient, as defined above, together with one or more acceptable carriers and optionally other therapeutic ingredients. The carrier(s) must be “acceptable” in the sense of being compatible with the other ingredients of the formulation and physiologically innocuous to the recipient thereof. The pharmaceutical formulations useful herein contain a pharmaceutically acceptable carrier, including any suitable diluent or excipient, which includes any pharmaceutical agent that does not itself induce the production of an immune response harmful to the vertebrate receiving the composition. As used herein, the term “pharmaceutically acceptable” also means being approved by a regulatory agency of the Federal or a state government or listed in the U.S. Pharmacopia, European Pharmacopia or other generally recognized pharmacopia for use in mammals, and more particularly in humans.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention also provides that the formulation be packaged in a hermetically sealed container such as an ampoule or sachette indicating the quantity of composition. In one embodiment, the composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the composition is supplied in liquid form in a hermetically sealed container indicating the quantity and concentration of the composition. Preferably, the liquid form of the composition is supplied in a hermetically sealed container.

Additional dosages can be administered, by the same or different route, to achieve the desired effect. Levels of induced TNF-α can be monitored, and dosages can be adjusted or repeated as necessary to elicit and maintain desired levels of effects.

Methods of administering a composition of the present invention include, but are not limited to, parenteral administration (e.g., intradermal, intramuscular, intravenous and subcutaneous), epidural, and mucosal (e.g., intranasal and oral or pulmonary routes or by suppositories). The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents.

The optimal therapeutically effective amount may be determined experimentally, taking into consideration the exact mode of administration, the form in which the drug is administered, the indication toward which the administration is directed, the subject involved (e.g., body weight, health, age, sex, etc.), and the preference and experience of the physician or veterinarian in charge.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the present invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl n-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The compounds of the present invention are agonists of N-formyl peptide receptors (FPR), particularly N-formyl peptide receptors like 1 (FPRL1). In one embodiment, the present invention provides a method of activating N-formyl peptide receptors (FPR) in a cell comprising contacting the cell with an effective amount of the compound of the present invention, or a salt, solvate, ester, and/or prodrug thereof.

As agonists of FPR, the present compounds can stimulate production of tumor necrosis factor α (TNF-α). In one embodiment, the present invention provides a method of stimulating production of tumor necrosis factor α (TNF-α) in a cell comprising contacting the cell with an effective amount of the compound of the present invention, or a salt, solvate, ester, and/or prodrug thereof.

In another embodiment, the present invention provides a method of inducing apoptosis in a tumor-associated cell comprising contacting the tumor-associated cell with an effective amount of the compound of claim 1, or a salt, solvate, ester, and/or prodrug thereof. In yet another embodiment, the present invention provides a method of treating a disease, condition, or symptom associated with TNF-α, such as a neoplastic disease, for a patient in need thereof comprising administering to the patient a therapeutically effective amount of the compound of the present invention, or a salt, solvate, ester, and/or prodrug thereof.

The present invention comprises development of immunomodulatory compounds/agents that enhance innate immune responses, which represents a promising strategy for combating infectious diseases. The inventors screened a series of 71 arylcarboxylic acid hydrazide derivatives for their ability to induce macrophage tumor necrosis factor alpha (TNF-α) production and identified 6 such compounds, including one compound previously shown to be a formyl peptide receptor (FPR/FPRL1) agonist. The two most potent compounds (Compound 1: nicotinic acid [5-3-bromophenyl)-2-furyl]methylene-hydrazide; Compound 2: 4-fluoro-benzoic acid [5-(3-trifluoromethyl-phenyl)-2-furyl]methylene-hydrazide) were selected for further analysis. These compounds induced de novo production TNF-α in a dose- and time-dependent manner in human and murine monocyte/macrophage cell lines and in primary macrophages. These compounds also induced mobilization of intracellular calcium, production of reactive oxygen species, and chemotaxis in human and murine phagocytes. Induction of macrophage TNF-α production was pertussis toxin-sensitive, and analysis of the cellular target of these compounds showed that they were FPRL1-specific agonists and that this response was blocked by FPR/FPRL1 and FPRL1-specific antagonists. Additionally, pharmacophore modeling showed a high degree of similarity for low-energy conformations of these two compounds to the current pharmacophore model for FPR ligands (Edwards et al., 2005). Overall, these compounds represent novel FPRL1 agonists that induce TNF-α, a response distinct from those induced by all other known FPR and FPRL1 agonists.

The innate immune system represents an essential first-line of defense against microbial pathogens and may also influence the nature of the subsequent adaptive immune response (Beutler, 2004; Hoebe et al., 2004). Phagocytic cells, such as macrophages and neutrophils, play a key role in innate immunity because of their ability to recognize, ingest, and destroy many pathogens by oxidative and non-oxidative mechanisms (Tosi, 2005). This response is modulated by a variety of extrinsic factors, including bacterial products, lipids, cytokines, and chemokines, and it is now apparent that the nature of a given inflammatory response represents an interplay between pro-inflammatory and anti-inflammatory immune modulators (reviewed in (Gordon, 2007)). Indeed, it has been suggested that immunomodulatory agents that enhance innate immune responses could represent a promising strategy to address current concerns of how to combat emerging diseases and drug-resistant infections (Schiller et al., 2006). Since one of the key factors regulating phagocyte inflammatory responses is TNF-α, the inventors screened a library of chemically-related compounds to identify unique small-molecule activators of phagocyte TNF-α production.

In primary screening, the inventors evaluated whether 26 previously characterized small-molecule phagocyte agonists (Schepetkin et al., 2007) induced phagocyte TNF-α production and identified two lead compounds with low-to-modest activity. Since these compounds were both arylcarboxylic acid hydrazides, the inventors used this chemical scaffold to select additional analogs for secondary screening. Four of these compounds were more active than the two parent compounds, and the two most potent arylcarboxylic acid hydrazides (Compounds 1 and 2) were selected for further characterization and analysis. Both of these compounds induced significant levels of TNF-α production by murine and human macrophages and macrophage cell lines. In addition, Compounds 1 and 2 activated [Ca²⁺+]ᵢ mobilization, chemotaxis, and ROS production in human and murine phagocytes. This array of neutrophil responses suggested that these compounds were possibly GPCR agonists (Zhelev and Alteraifi, 2002). Indeed, the inventors' previous study showed that Compound AG-14 was an FPR/FPRL1 agonist (Schepetkin et al., 2007). Additionally, PTX treatment significantly inhibited TNF-α production by cells treated with either of these compounds, which is consistent with GPCR signaling. Thus, the inventors considered whether Compounds 1 and 2 might also be FPR/FPRL1 agonists, albeit unique because none of the known FPR/FPRL1 agonists have been shown to induce TNF-α production and neither fMLF, nor WKYMVm, was able to induce macrophage TNF-α production. In fact, it has been reported that fMLP can actually inhibit TNF-α production by LPS-stimulated neutrophils (Vulcano et al., 1998).

To directly evaluate whether Compounds 1 and 2 were FPR/FPRL1 agonists, the inventors tested these compounds for their ability to induce [Ca²⁺]ᵢ mobilization in cells expressing either FPR or FPRL1. Note that all potent TNF-α inducers activated [Ca²⁺]ᵢ mobilization in human neutrophils; however, not all arylcarboxylic acid hydrazides that induced [Ca²⁺]ᵢ mobilization were TNF-α inducers (see Table 1). Interestingly, Compounds 1 and 2 dose dependently activated [Ca²⁺]ᵢ mobilization only in cells expression FPRL1, but not in wild-type cells or cells expressing FPR. In contrast, Compound AG-14 activated [Ca²⁺]ᵢ mobilization only in cells expressing FPR, which may help to explain the differences in responses induced by this compound versus Compounds 1 and 2. Thus, although these three compounds are all arylcarboxylic acid hydrazides, their receptor specificity is clearly distinct, resulting in receptor subtype specificity. Furthermore, a general FPR/FPRL1 antagonist and a specific FPRL1 antagonist were both found to block TNF-α induction by Compounds 1 and 2, providing direct evidence that this response was mediated through activation of FPRL1. Note, however, that the inventors have not performed an analysis of all possible GPCRs and cannot rule out the possibility that additional GPCRs might be involved in the responses observed. Since neither of the three compounds induced [Ca²⁺+]ᵢ mobilization in wild-type RBL cells, the inventors' results do indicate that these agonists do not activate other endogenous GPCRs in RBL cells.

Pharmacophore modeling is based on the premise that all ligands of a given target bind in a conformation that presents similar steric and electrostatic features to the target receptor, and these features are recognized by the receptor and are responsible for biological activity (Guner et al., 2004). The pharmacophore model used here was developed based on the bovine rhodopsin crystal structure and known FPR ligands (Edwards et al., 2005). The inventors' molecular modeling showed a high degree of similarity for low-energy conformations of Compounds 1 and 2 to this previously published pharmacophore model, demonstrating a good agreement between biological activity and the presence of low-energy, best-fit conformations of the compounds with the model. Although the inventors' previous conformational analysis showed that one FPRL1 agonist, Quin C1, did not fit well into the pharmacophore model (Schepetkin et al., 2007), the present results indicate that the model is not specific only for FPR ligands and that some FPRL1 agonists also fit into the model. Overall, pharmacophore modeling provided further support that these compounds do indeed fit the molecular features required for FPR/FPRL1 agonists. Currently, there is very little information on the structure of FPRL1. Thus, the inventors suggest that the arylcarboxylic acid hydrazides reported here and additional analogs may be exploited in the future to probe the requirements for ligand interaction and receptor activation.

Collectively, the inventors' data support the conclusion that Compounds 1 and 2 activate PTXsensitive FPRL1 to induce intracellular signaling events that induce a number of host defense/inflammatory responses, including the stimulation of TNF-α production in macrophages. Note, however, these compounds must activate distinct signaling cascades, since the standard peptide agonists of FPR/FPRL1 failed to induce TNF-α production, although these agonists are known to activate many of the other cellular responses observed ([Ca²⁺]ᵢ mobilization, ROS production, chemotaxis). Previously, various natural and synthetic small-molecules have been shown to induce macrophage TNF-α production, which has been shown to be involved in the immunomodulatory properties of these drugs, such as taxol and imiquimod (Wagner et al. 1997; Byrd-Leifer et al., 2001; Kirikae et al., 2000). However, all non-peptide synthetic substances known to activate macrophage TNF-α production have been shown to be Toll-like receptor (TLR)-4, -7, and -8 agonists (Byrd-Leifer et al., 2001; Wang et al., 2002; Schön and Schön, 2007a). Thus, the small-molecule compounds described here are unique in their ability to activate this response via a GPCR. In addition, these compounds represent a novel chemotype, and no other arylcarboxylic acid hydrazides have been shown to be potent TNF-α inducers or agonists of GPCR, including FPR/FPRL1. On the other hand, compounds with this scaffold have been reported among agents with antitumor (Boykin, Jr. and Varma, 1970), proapoptotic (Zhang et al., 2004), antiprion (Bertsch et al., 2005), analgesic (Almasirad et al., 2006), and antioxidant (Hermes-Lima et al., 1998; Simunek et al., 2005) properties. Thus, the inventors' findings suggest the possibility that the antitumor and proapoptotic effects reported for several related hydrazides (Boykin, Jr. and Varma, 1970; Zhang et al., 2004) could be due, in part, to activation of TNF-α production. Indeed, FPR/FPRL1 has been reported to be expressed on human tumor cells, including malignant human glioma cell lines, primary glioblastomas and anaplastic astrocytomas, and epithelial cancer cells, and it has been suggested that this receptor contributes to motility, growth, and angiogenesis (Le et al., 2000; Rescher et al., 2002; Zhou et al., 2005; Huang et al., 2007). In addition, transduction of human FPRL1 into murine tumor cells has been reported to increase host anti-tumor immunity, although this was not due to an antibody response to the foreign antigen, as anti-FPRL1 antibodies were not found (Hu et al., 2005). One possibility suggested by the present studies is that expression of FPRL1 in these tumor cells could result in responsiveness to endogenous agonists that induce TNF-α production, which would lead to tumor cell apoptosis and destruction (Lejeune et al., 2006; Reed, 2006).

FPRL1 is a highly promiscuous receptor and responds to a wide array of exogenous and endogenous ligands, including formyl peptides, non-formylated synthetic peptides, lipoxin A4, serum amyloid A, β-amyloid peptide Aβ1-42, prion peptide PrP1, annexin 1, small-molecule agonists, and others (reviewed in (Migeotte et al., 2006). Likewise, the array of responses induced by these ligands is varied, and the intracellular signals that are activated depend on the ligand, ligand concentration, and cellular features. For example, stimulation of FPRL1 with lipoxin A4 and/or annxexin 1/annexin 1-derived peptides leads to anti-inflammatory responses (Perretti et al., 2002; Serhan, 2007). Conversely, inflammatory responses are induced by a number of the other FPRL1 agonists, including cathelicidin antibacterial peptide LL37 (Yang et al., 2000), the truncated chemokine, sCKβ8-1 (Elagoz et al., 2004), and pituitary adenylate cyclase-activating polypeptide 27 (Kim et al., 2006). Here, the inventors describe an additional class of FPRL1 agonists that induce a response not seen before via FPRL1 activation. However, given the wide range of responses induced by FPRL1 agonists so far, it is not surprising that additional responses, such as the induction of TNF-α production shown here, will eventually be identified as novel ligands for this receptor are found. The wide range of agonists and responses of FPRL1 suggests that this receptor may represent a unique target for therapeutic drug design. Here, the inventors have identified and characterized specific FPR and FPRL1 agonists that not only have a novel in structure (arylcarboxylic acid hydrazides) but also induce a novel response not seen before with any of the known FPRL1 agonists. Thus, further development of this class of agonists and analysis of additional derivatives may provide important clues to understanding FPRL1 and may lead to the identification of small-molecule agonists with even higher efficacy. In addition, such small molecule inducers of TNF-α production may also have potential as anticancer therapeutics.

The inventors utilized a similar approach for computational SAR analysis of a large group of arylcarboxylic acid hydrazides, including our previously reported derivatives 8 and several novel analogs identified here in further screening. These studies provide further optimization of these molecules as lead compounds that can induce macrophage TNF-α production and also provide clues to the molecular features required for agonist activity.

The present invention is further illustrated by the following examples that should not be construed as limiting. The contents of all references, patents, and published patent applications cited throughout this application, as well as the Figures, are incorporated herein by reference in their entirety for all purposes.

### EXAMPLE

### Example 1

### Materials and Methods of the Present Invention

### Materials

All synthetic small-molecule compounds were obtained from TimTec Inc. (Newark, Del.). 8-Amino-5-chloro-7-phenylpyridol [3,4-d]pyridazine-1,4(2H,3H)-dione (L-012) was obtained from Wako Chemicals (Richmond, Va.). Actinomycin D (ActD), cycloheximide (CHX), concanavalin A, dimethyl sulfoxide (DMSO), ethylenediaminetetraacetic acid (EDTA), ionomycin, horseradish peroxidase (HRP), N-formyl-Met-Leu-Phe (fMLF), Percoll, 4-(2-hydroxyethyl)-1-piperazineethanesulfonic acid (HEPES), Histopaque 1077, Histopaque 1119, lipopolysaccharide (LPS) from Escherichia coli K-235, and phorbol 12-myristate 13-acetate (PMA) were purchased from Sigma Chemical Co. (St. Louis, Mo.). IL-8 and keratinocytederived chemokine (KC) were obtained from PeproTech Inc. (Rocky Hill, N.J.). Pertussis toxin (PTX), WKYMVm, and human GM-CSF were purchased from Calbiochem (San Diego, Calif.). Fura-2AM was from Molecular Probes (Eugene, Oreg.). The receptor antagonists N-tbutoxycarbonyl-Phe-Leu-Phe-Leu-Phe (Boc-2) and WRWWWW (WRW4) were obtained from Phoenix Pharmaceuticals (Belmont, Calif.). Dulbecco's Modified Eagle's Medium (DMEM) and Hanks' balanced salt solutions (10×), pH 7.4 (10×HBSS) (without phenol red, with and without Ca²⁺ and Mg²⁺, HBSS⁺ and HBSS⁻, respectively), and enzyme-free cell dissociation buffer were from Invitrogen (Carlsbad, Calif.). RPMI 1640 media was purchased from Mediatech (Herdon, Va.). Percoll stock solution was prepared by mixing Percoll with 10×HBSS at a ratio of 9:1.

### Cell Culture

Murine macrophage J774.A1 cells were cultured in DMEM supplemented with 10% (v/v) heat-inactivated fetal bovine serum (FBS), 10 mM HEPES, 100 μg/ml streptomycin, and 100 U/ml penicillin. Cells were grown to confluence in sterile tissue culture flasks and gently detached by scraping. Human monocyte/macrophage MonoMac6 cells (DSMZ, Germany) were cultured in RPMI 1640 supplemented with 10% (v/v) FBS, 10 μg/ml bovine insulin, 100 μg/ml streptomycin, and 100 U/ml penicillin. Rat basophilic leukemia (RBL-2H3) cells transfected with human FPR (RBL-FPR) or FPRL1 (RBL-FPRL1) were cultured in DMEM supplemented with 20% (v/v) FBS, 10 mM HEPES, 100 μg/ml streptomycin, and 100 U/ml penicillin, as described previously (Nanamori et al., 2004). Human monocytic THP-1 cells were cultured in RPMI 1640 medium supplemented with 10% (v/v) FBS, 100 μg/ml streptomycin, and 100 U/ml penicillin. All cell cultures were grown at 37° C. in a humidified atmosphere containing 5% CO2. Cell number and viability were assessed microscopically using trypan blue exclusion.

### Isolation of Murine Bone Marrow Neutrophils and Peritoneal Macrophages

All animal use was conducted in accordance with a protocol approved by the Institutional Animal Care and Use Committee at Montana State University. Bone marrow leukocytes were flushed from tibias and femurs of BALB/c mice with HBSS− supplemented with 0.1% BSA and 1% glucose and the neutrophils were purified on Percoll density gradients, as previously described (Schepetkin et al., 2007). Peritoneal macrophages were isolated from BALB/c mice 4 days after intraperitoneal injection of 1 ml saline containing 100 μg concanavalin A, as described previously (Schepetkin et al., 2005). Peritoneal exudate cells were suspended in RPMI 1640 containing 10% (v/v) FBS with antibiotics (penicillin and streptomycin) and incubated at 37° C. in 100 mm tissue culture dishes for 1 h. Adherent peritoneal macrophages were harvested and cultured with the compounds under investigation for 24 hr at 37° C. and 5% CO2.

### Isolation of Human Neutrophils and Monocyte/Macrophages

Blood was collected from healthy donors in accordance with a protocol approved by the Institutional Review Board at Montana State University. Neutrophils were purified from the blood using dextran sedimentation, followed by Histopaque 1077 gradient separation and hypotonic lysis of red blood cells, as described previously (Gauss et al., 2005; Gauss et al., 2007). Isolated neutrophils were washed twice and resuspended in HBSS−. Neutrophil preparations were routinely >95% pure, as determined by light microscopy, and >98% viable, as determined by trypan blue exclusion. Monocytes were isolated from blood using dextran sedimentation, Histopaque 1077 gradient separation. Adherent monocytes were cultured for 7 days in RPMI-1640 containing 10% FBS and 50 ng/ml GM-CSF to induce macrophage differentiation.

### Ca²⁺ Mobilization Assay

Changes in intracellular Ca²⁺ were measured with a FlexStation II scanning fluorometer using a FLIPR 3 Calcium Assay Kit (Molecular Devices, Sunnyvale, Calif.) for human neutrophils and RBL-FPR and RBL-FPRL1 cells or fluorescent dye Fura-2AM for J774.A1 macrophages. Human neutrophils or transfected RBL cells, suspended in HBSS− containing 10 mM HEPES were loaded with FLIPR Calcium 3 dye following the manufacturer's protocol. After dye loading, Ca²⁺ was added to the cell suspension (2.25 mM final), and 100 μl of cell suspension were aliquoted into the wells of a flat-bottom black microtiter plate (2×10⁵ cells/well). The compound source plate contained dilutions of test compounds in HBSS+. Changes in fluorescence were monitored (ex=485 nm, em=525 nm) every 5 sec for 240 to 500 sec at room temperature after automated addition of compounds to the cells. Maximum change in fluorescence, expressed in arbitrary units over baseline, was used to determine agonist response. Curve fitting and calculation of median effective concentration values (EC₅₀) were performed by nonlinear regression analysis of the dose-response curves generated using Prism 5 (GraphPad Software, Inc., San Diego, Calif.). J774A.1 macrophages suspended in HBSS− containing 10 mM HEPES were loaded with Fura-2AM dye (2 μg/ml final concentration) and incubated for 30 minutes in dark at 37° C. After dye loading, the cells were washed with HBSS− containing 10 mM HEPES, resuspended in 1.5 ml of HBSS+ containing 10 mM HEPES, and aliquoted into the wells of a flat clear-bottom, halfarea-well black microtiter plate (6×10⁵ cells/well). The compound source plate contained dilutions of test compounds in HBSS+. Changes in fluorescence were monitored (ex=340 nm and 380 nm, em=510 nm) every 5 sec for 240 to 500 sec at 37° C. after automated addition of compounds to the wells. Maximum change in ratio of fluorescence values at excitation wavelengths of 340 and 380 nm was used to determine agonist response. Curve fitting and calculations were performed as above.

### Chemotaxis Assay

Neutrophils were suspended in HBSS+ containing 2% (v/v) FBS (2×10⁶ cells/ml), and chemotaxis was analyzed in 96-well ChemoTx chemotaxis chambers (Neuroprobe, Gaithersburg, Md.), as described previously (Schepetkin et al., 2007). Briefly, lower wells were loaded with 30 μl of HBSS+ containing 2% (v/v) FBS and the indicated concentrations of test compound, DMSO (negative control), 50 nM IL-8 as a positive control for human neutrophils, or 50 nM keratinocyte-derived chemokine (KC) as a positive control for murine neutrophils. The number of migrated cells was determined by measuring ATP in lysates of transmigrated cells using a luminescence-based assay (CellTiter-Glo; Promega, Madison, Wis.), and luminescence measurements were converted to absolute cell numbers by comparison of the values with standard curves obtained with of known numbers of neutrophils. The results are expressed as percentage of negative control and were calculated as follows: (number of cells migrating in response to test compounds/spontaneous cell migration in response to control medium)×100. EC₅₀ values were determined by nonlinear regression analysis of the dose response curves generated using Prism 5 software.

### Analysis of ROS Production

J774.A1 macrophages were plated at density of 1.5×10⁵ cells/well in wells of 96-well flat-bottom white microtiter plates (Corning Incorporated, Costar). After 16 hr (5% CO2, 37° C.), the media was aspirated, and test compounds or vehicle (final DMSO concentration of 1%) were added in fresh media (DMEM without phenol red, supplemented with 3% (v/v) FBS). After 5 min, the media was replaced by HBSS+ containing 4 μM L-012 and 8 μg/ml HRP, and luminescence was monitored for 120 min (2-min intervals) at 37° C. using a Fluoroscan Ascent FL microtiter plate reader (Thermo Electron, Waltham, Mass.). The curve of light intensity (in relative luminescence units) was plotted against time, and the area under the curve was calculated as total luminescence. The percent activation of ROS was calculated as follows: % activation=(sample-DMSO control)/DMSO control×100. The minimal compound concentration that enhanced ROS production by 50% above background control cells (AC50) was determined by graphing the % activation of ROS vs. log [test compound]. Each curve was determined using five to seven compound concentrations. Human neutrophils were plated at density of 5×10⁵ cells/well in wells of 96-well flatbottom white microtiter plates, and compounds under investigation or vehicle (final DMSO concentration of 1%) were added. After 5 min, the media was replaced by HBSS+ containing 40 μLM L-012, and luminescence was monitored for 120 min (2-min intervals) at 37° C. using a Fluoroscan Ascent FL microtiter plate reader. The results were analysed as described above to determine % activation and AC50 values.

### Determination of TNF-α and IL-6

Cells were treated for 24 hr with DMSO control, test compound, or LPS and mouse TNF-α or IL-6 and human TNF-α enzyme-linked immunosorbent assay (ELISA) kits (BD Biosciences Pharmigen) were used to detect these cytokines in the cell supernatants. Cytokine concentration was determined by extrapolation from the TNF-α/IL-6 standard curve, according to the manufacturer's protocol.

For treatments, cells were plated in 96-well microtiter plates at 2×105 cells/well (J774.A1, MonoMac6) or 6×104 cells/well (human monocyte-derived macrophages) using their respective culture media, except FBS was reduced to 3% (v/v). After incubation overnight (5% CO2, 37° C.), the media were removed and replaced with fresh media containing test compounds.

### Cytotoxicity Assay

Cytotoxicity was analyzed with a CellTiter-Glo Luminescent Cell Viability Assay Kit (Promega, Inc., Madison, Wis.), according to the manufacturer's protocol. Briefly, J774.A1 cells were cultured at a density of 3×104 cells/well with the test compounds for 24 hr at 37° C. and 5% CO2, substrate was added, and luminescence signal in the samples was analyzed with a Fluoroscan Ascent FL.

### Endotoxin Assay

Endotoxin was measured using Limulus Amebocyte Lysate Pyrogent Plus (Cambrex Bio Science, Walkersville, Md.). Briefly, the limulus amebocyte lysate was reconstituted in 250 μl solution of test compound (50 μM in endotoxin free water/1% DMSO), and each vial was incubated at 37° C. for 1 hr. At the end of the incubation period, each vial was inverted 180° to estimate gel formation in comparison with control (endotoxin free water).

### Conformational Analysis

For the selected compounds, sets of conformations were generated using the Conformational Search Module, as implemented in HyperChem Version 7.0 (Hypercube, Inc., Waterloo, ON, Canada). The systematic search of conformations for each compound was performed by energy minimization, starting with 2000 initial geometries at random values of torsion angles about exocyclic single bonds and considering that hydrazide C═N fragment can adopt syn- or anti-orientation of substituents. Energy was minimized by the Polak-Ribiere conjugate gradient method with MM+ force field (HyperChem). Attainment of a root-mean-square gradient <0.02 kcal/mol/Å was used as the termination condition for minimization. Conformations were considered equivalent if all the corresponding torsion angles differed by less than 20° among conformations. Results of the conformational search were saved as text files in HCS format adopted in HyperChem. These files were used as input for a computer program that analyzed each conformation and calculated distances between atoms specified by user as potential acceptors of H-bonds or hydrophobic points. We placed the hydrophobic centers in the middle of the benzene rings in Compound 2 and in the geometric gravity center of the bromophenyl group in Compound 1; whereas, the acceptors were positioned at fluorine atoms, pyridine and doublebonded hydrazide nitrogens, as well as at carbonyl and furan oxygen atoms, which are known as H-bond acceptors. The distances calculated for all possible triads of the two H-bond acceptors and single hydrophobic center among all the conformations found were compared with those in the pharmacophore model published previously by Edwards et al. (Edwards et al., 2005). The value of ε was calculated as root-mean-square relative deviation of the distances obtained from the center of corresponding tolerance intervals [A1-A2 (3-6 Å), A1-H (5-7 Å), and A2-H (4-7 Å)], as determined by Edwards et al. (Edwards et al., 2005).

### Structure Encoding by Atom Pairs

For the purpose of SAR analysis, we used an atom pair representation of molecular structures with each atom pair denoted as T1_D_T2, where T1 and T2 are the types of atoms in the pair and D is the topological (bond) distance or number of bonds in the shortest path between these atoms in the structural formula. As previously reported (Khlebnikov et al., 2008), T1 and T2 were defined with symbolic codes used in HyperChem, Version 7 (Hypercube, Inc., Gainesville, Fla.) for atom type representation within MM+ force field. For example, CA, CO, and C3 codes were used for sp2-hybridized aromatic, carbonyl, and furan carbon atoms, respectively. This approach allows easy generation of atom pairs directly from the output file containing the molecular structure (HIN file) built by HyperChem. As atom pairs T1_D_T2 and T2_D_T1 are equivalent, we used a unified definition with lexicographic order of type substrings (i.e., with T1≦T2).

All 836 unique atom pairs possible for non-hydrogen atoms in the 86 derivatives of arylcarboxylic acid hydrazides were generated. This 86×836 data matrix was automatically built by the inventors' CHAIN program, based on HIN files created in HyperChem. A matrix element at the intersection of the ith row and jth column was equal to the jth atom pair occurrence in the ith molecule.

### Derivation of SAR Classification

Derivation of SAR classification was performed first by the LDA method with the “Forward Stepwise” option, using the corresponding module of STATISTICA 6.0. Statistical criterion for inclusion or exclusion of descriptors at each step was p≦0.05. The stepwise LDA allowed selection of 14 significant descriptors from 836 atom pairs generated initially. The LDA run was then repeated with the “Best Subset Search” option on the basis of 14 variables selected in the first LDA run. The best subset consisted of 13 atom pairs giving the least misclassification error of LDA model. Starting from 13 variables of the best subset, we developed binary classification tree models with discriminant-based linear combination splits (CTLCS) and with univariate splits. The classification trees were built with STATISTICA 6.0 using estimated prior probabilities and equal misclassification costs for classes (Loh et al., 1997; Breiman et al., 1984). An exhaustive C&RT-style univariate split selection method was used, as described by Breiman et al. (Breiman et al., 1984).

### Example 2

### Identification of TNF-α Inducer

Previously, the present inventors screened a chemolibrary of drug-like molecules for their ability to activate reactive oxygen species (ROS) production by phagocytes and identified 26 such agonists (designated here as agonists (AG) 1-26, see Schepetkin et al., 2007).

The present inventors evaluated whether these compounds also induced phagocyte TNF-α production and found that five compounds induced modest levels of TNF-α production by murine J774.A1 macrophages (Compounds AG-1, -3, -8, -14, and -15 in Table 1). Since the two most potent compounds (AG-3 and AG-14 in Table 1) are both arylcarboxylic acid hydrazides (see Table 1), the inventors focused on this scaffold and selected analogs for each of these two agonists for further screening.

TABLE 1
Effect of Arylcarboxylic Acid Hydrazide Derivatives on TNF-α
Production in J774.A1 Macrophages and Ca²⁺ Mobilization in Human Neutrophils

| | | | [Ca²⁺]i | |
|---|---|---|---|---|
| | | TNF-α | EC₅₀ | |
| Number | Structure | production^{a} | (μM) | LogP^{c} |
| 1 | 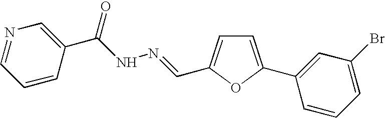 | 40-50 | 0.3 | 2.883 |
| 2 | 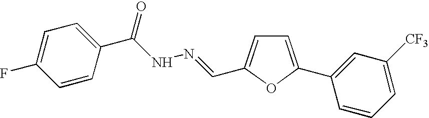 | 30-40 | 0.6 | 4.974 |
| 3 | 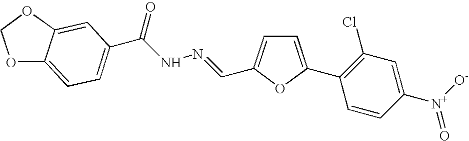 | 30-40 | 0.3 | 3.998 |
| 4 | 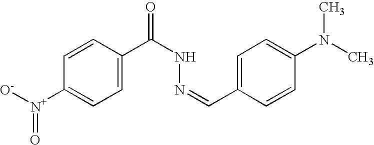 | 20-25 | 9 | 2.357 |
| 5 | 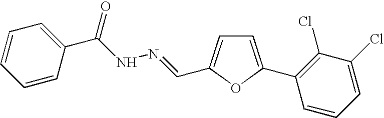 | 7-10 | 2.3 | 4.616 |
| 6 (AG-14) | 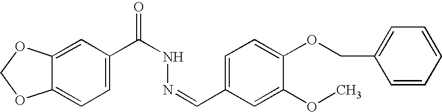 | 5-8 | 0.63 | 4.189 |
| 7 | 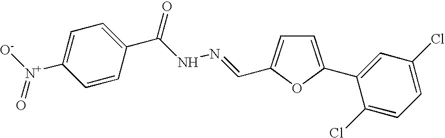 | <5 | 10 | 4.296 |
| 8 | 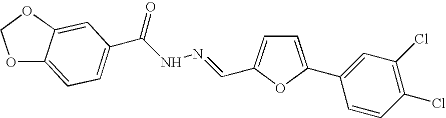 | <5 | 11.9 | 4.82 |
| 9 (AG-3) | 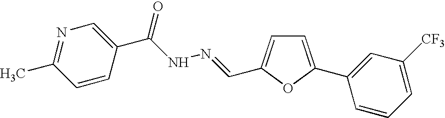 | <5 | 15.8 | 3.721 |
| 10 | 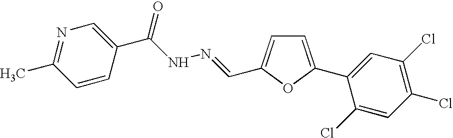 | <5 | 32 | 4.094 |
| 11 (AG-105) | 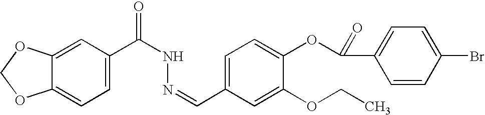 | <5 | N.A.^{b} | 5.823 |
| 12 ((AG-108) | 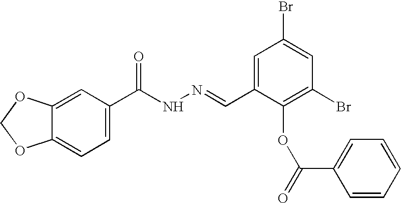 | <5 | N.A. | 5.212 |
| 13 | 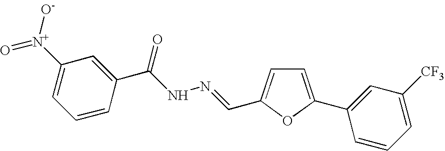 | <5 | N.A. | 4.664 |
| 14 | 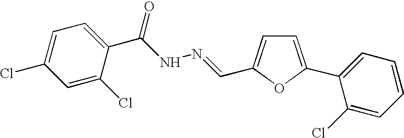 | <5 | N.A. | 5.125 |
| 15 | 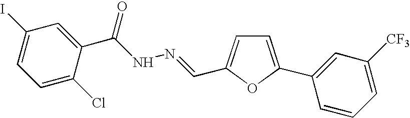 | <5 | N.A. | 6.088 |
| 16 | 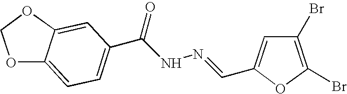 | <5 | N.A. | 3.461 |
| 17 | 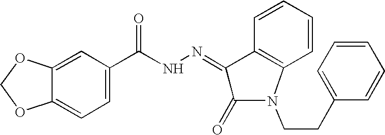 | <2 | 8.9 | 4.633 |
| 18 (AG-104) | 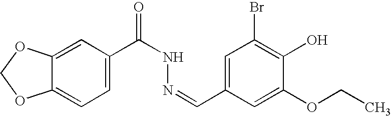 | <2 | 12.2 | 3.621 |
| 19 | 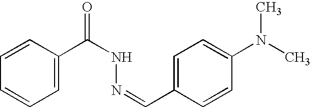 | <2 | 14.5 | 2.73 |
| 20 | 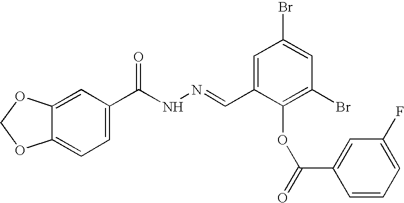 | <2 | 18.9 | 5.465 |
| 21 | 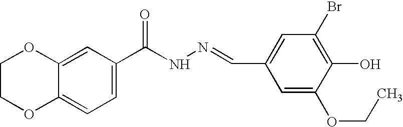 | <2 | 21.9 | 3.378 |
| 22 | 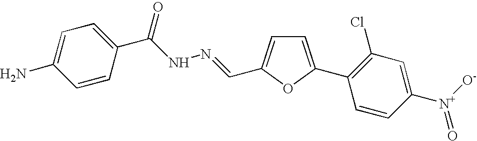 | <2 | N.A. | 2.595 |
| 23 | 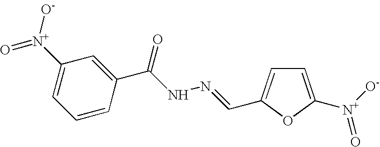 | <2 | N.A. | 1.12 |
| 24 | 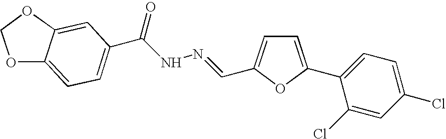 | <2 | N.A. | 4.87 |
| 25 | 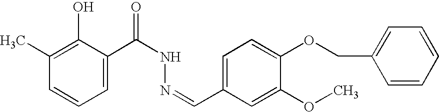 | N.A. | 0.74 | 5.683 |
| 26 | 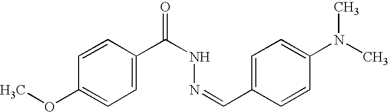 | N.A. | 3 | 2.599 |
| 27 | 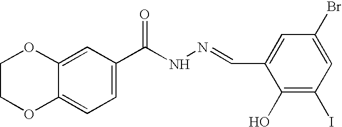 | N.A. | 8 | 6.011 |
| 28 | 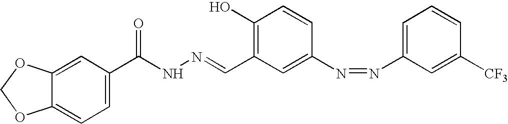 | N.A. | 8.5 | 7.005 |
| 29 | 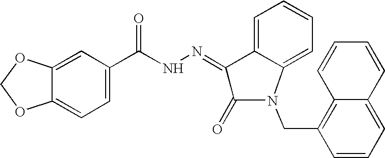 | N.A. | 9.3 | 5.448 |
| 30 | 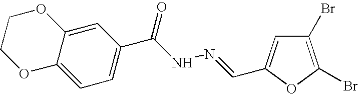 | N.A. | 9.8 | 3.218 |
| 31 | 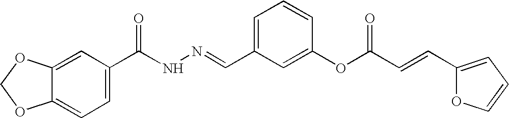 | N.A. | 12.4 | 4.319 |
| 32 | 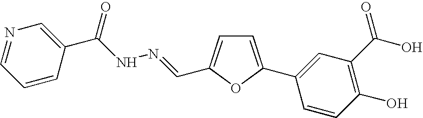 | N.A. | 12.5 | 2.193 |
| 33 | 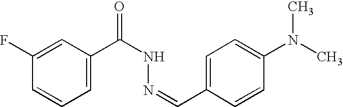 | N.A. | 13.1 | 2.606 |
| 34 | 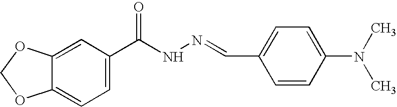 | N.A. | 17.9 | 2.842 |
| 35 | 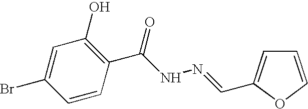 | N.A. | 22.7 | 4.092 |
| 36 | 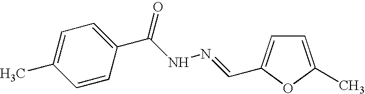 | N.A. | 25.5 | 2.549 |
| 37 | 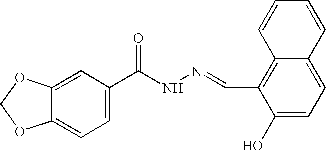 | N.A. | 26.8 | 5.048 |
| 38 | 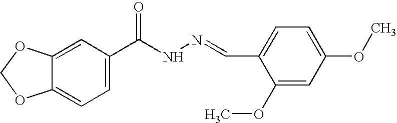 | N.A. | 28.1 | 2.146 |
| 39 | 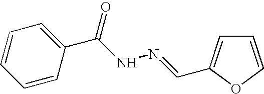 | N.A. | 28.4 | 1.629 |
| 40 (AG-106) | 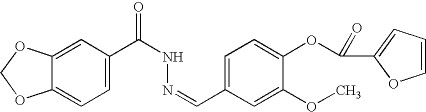 | N.A. | N.A. | 3.512 |
| 41 (AG-107) | 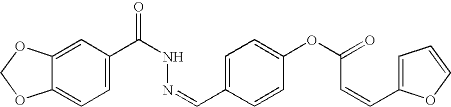 | N.A. | N.A. | 4.357 |
| 42 (AG-109) | 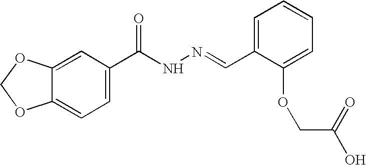 | N.A. | N.A. | 1.604 |
| 43 | 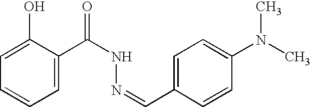 | N.A. | N.A. | 3.876 |
| 44 | 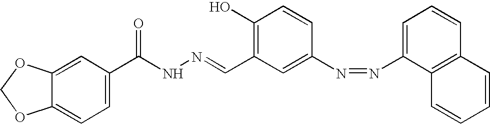 | N.A. | N.A. | 6.866 |
| 45 | 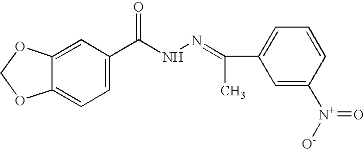 | N.A. | N.A. | 3.004 |
| 46 | 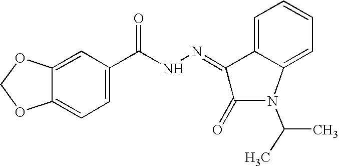 | N.A. | N.A. | 3.319 |
| 47 | 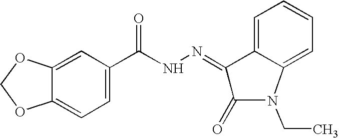 | N.A. | N.A. | 2.972 |
| 48 | 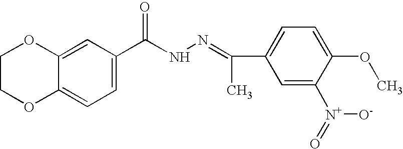 | N.A. | N.A. | 3.091 |
| 49 | 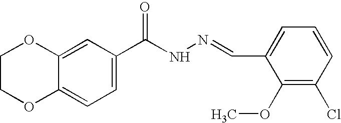 | N.A. | N.A. | 2.977 |
| 50 | 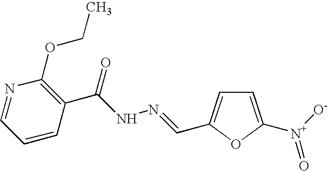 | N.A. | N.A. | 0.661 |
| 51 | 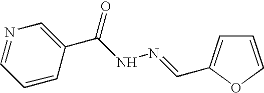 | N.A. | N.A. | 0.137 |
| 52 | 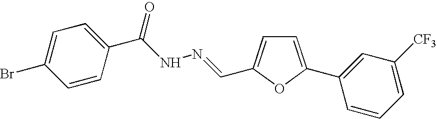 | N.A. | N.A. | 5.694 |
| 53 | 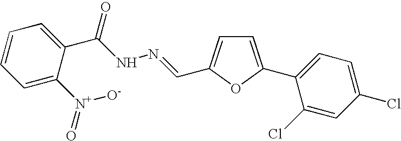 | N.A. | N.A. | 4.255 |
| 54 | 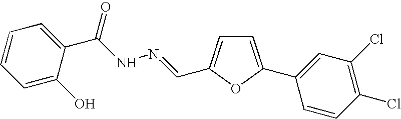 | N.A. | N.A. | 5.854 |
| 55 | 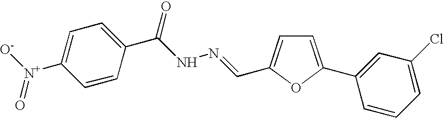 | N.A. | N.A. | 3.89 |
| 56 | 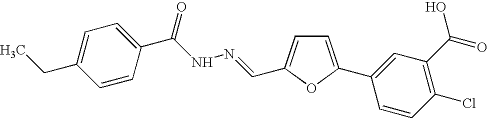 | N.A. | N.A. | 3.547 |
| 57 | 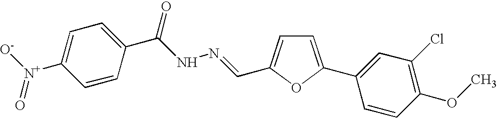 | N.A. | N.A. | 3.655 |
| 58 | 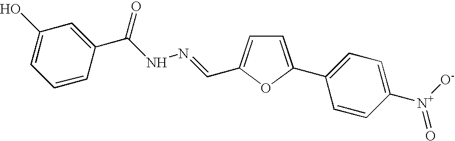 | N.A. | N.A. | 3.122 |
| 59 | 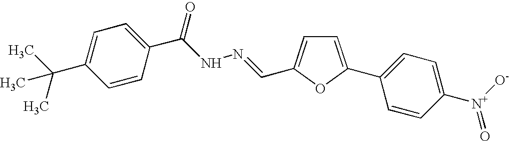 | N.A. | N.A. | 5.002 |
| 60 | 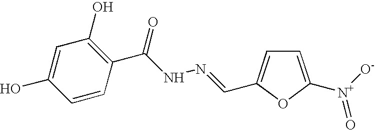 | N.A. | N.A. | 1.749 |
| 61 | 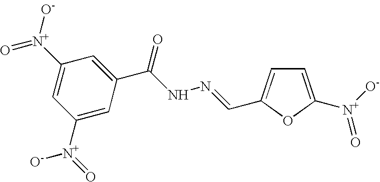 | N.A. | N.A. | 2.97 |
| 62 | 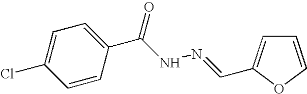 | N.A. | N.A. | 2.393 |
| 63 | 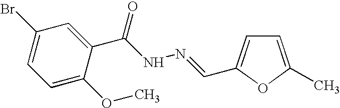 | N.A. | N.A. | 2.906 |
| 64 | 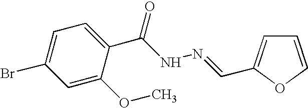 | N.A. | N.A. | 2.42 |
| 65 | 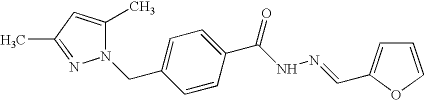 | N.A. | N.A. | 2.111 |
| 66 | 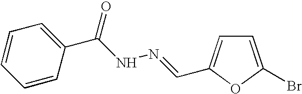 | N.A. | N.A. | 2.626 |
| 67 | 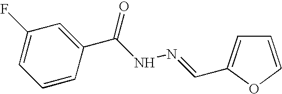 | N.A. | N.A. | 4.978 |
| 68 | 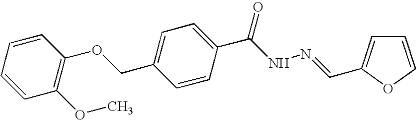 | N.A. | N.A. | 3.021 |
| 69 | 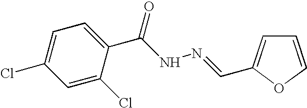 | N.A. | N.A. | 3.092 |
| 70 | 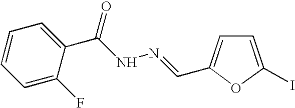 | N.A. | N.A. | 2.49 |
| 71 | 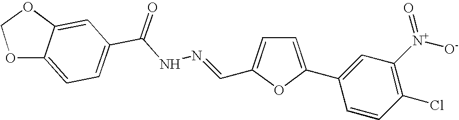 | N.A. | N.A. | 3.789 |

^{a}TNF-αproduction is presented as the relative fold of activation compared to vehicle (DMSO) control for 50 μM of the indicated compound.
^{b}N.A., no activity was observed.
^{c}LogP is the octanol/water partition coefficient. ACD/LogP values were obtained from www.emolecules.com.

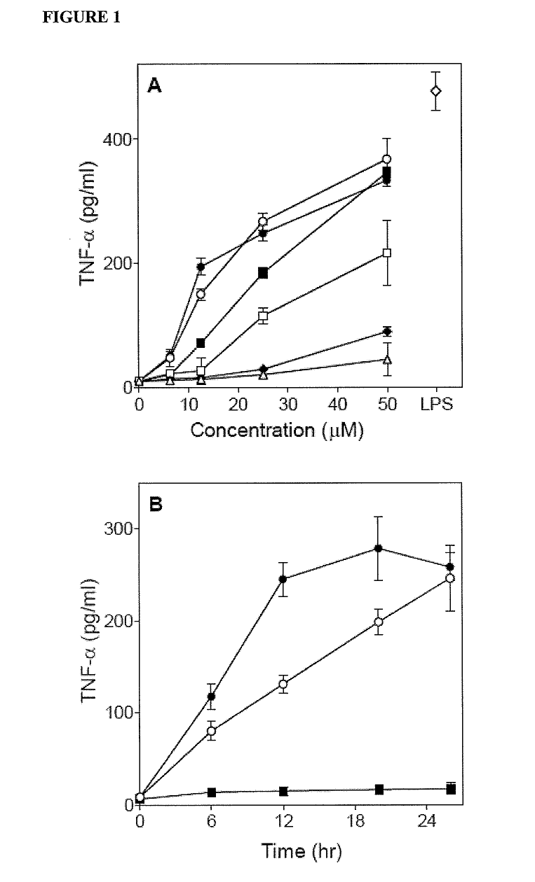

Forty-one analogs of AG-3 and 28 derivatives of AG-14 were selected to obtain a pool of 71 compounds for secondary screening (see Schepetkin et al., 2008). Analysis of these analogs for their ability to stimulate TNF-α production identified 22 additional compounds with various levels of activity (Table 1 and Table 2). Determination of dose-response relationships for all 24 selected compounds showed that six compounds were quite active and induced TNF-α production by J774.A1 macrophages in a concentration-dependent manner (see Table 2 and FIG. 1A).

TABLE 2
Chemical structures of the most potent TNF-α inducers

| Compound Number | Structure |
|---|---|
| 1 | 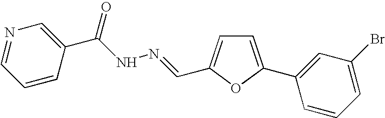 |
| 2 | 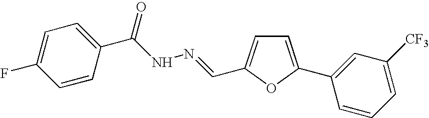 |
| 3 | 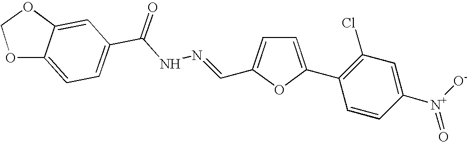 |
| 4 | 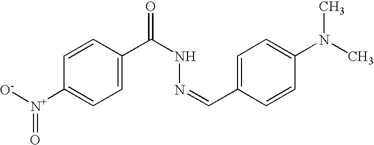 |
| 5 | 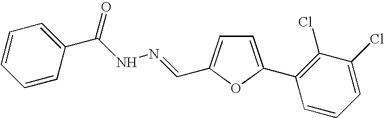 |
| 6 (AG-14) | 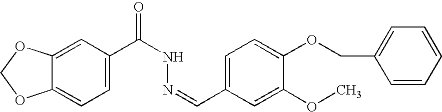 |

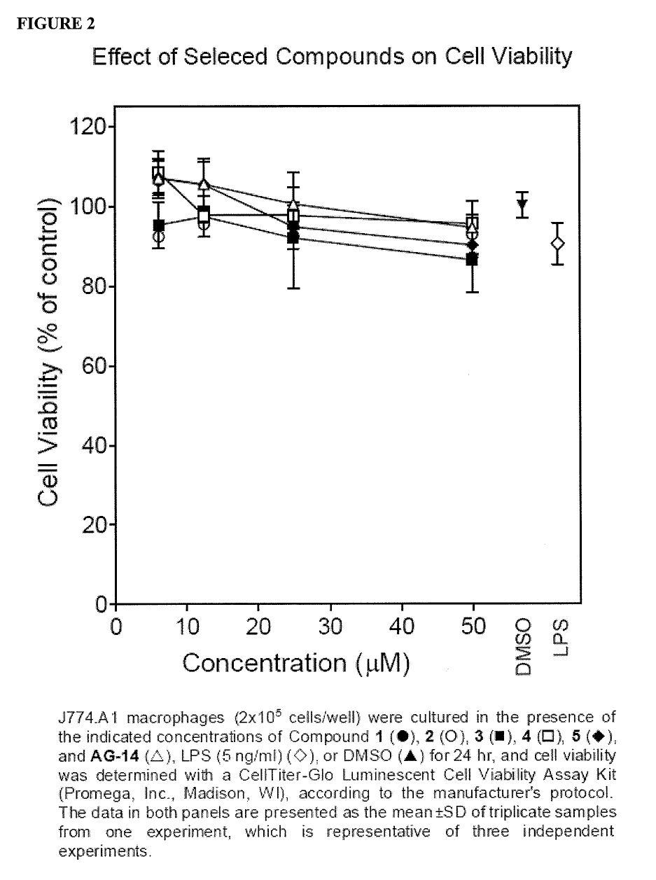

Compounds 1 and 2 in Table 2 were the most active, inducing 70-80% of the TNF-α levels that were induced by LPS. Activation was not due to LPS contamination, however, as analysis of Compounds 1 and 2 for possible LPS contamination using a limulus amebocyte lysate assay showed that these compounds contained no endotoxin (below detection limit; data not shown). TNF-α production was induced in a time-dependent manner by Compounds 1 and 2 (FIG. 1B); whereas, treatment with these compounds had no effect on cell viability, indicating lack of cytotoxicity (FIG. 2). Thus, further studies focused primarily on Compounds 1 and 2.

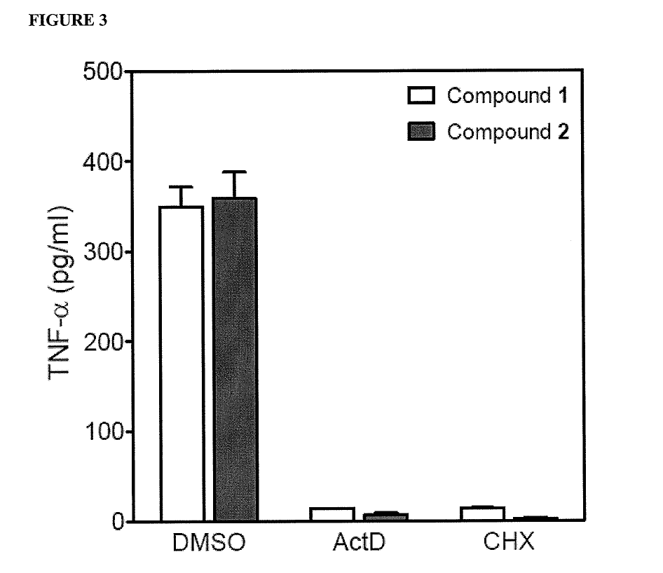

To determine whether the up-regulation of TNF-α production represented new protein synthesis, J774.A1 macrophages were treated with actinomycin D (ActD) to inhibit transcription or cycloheximide (CHX) to inhibit protein synthesis prior to treatment with active compounds. As shown in FIG. 3, treatment with either ActD or CHX completely inhibited TNF-α production induced by Compounds 1 and 2. These results indicate that transcription and de novo protein synthesis are required for the TNF-α secretion induced by these arylcarboxylic acid hydrazides.

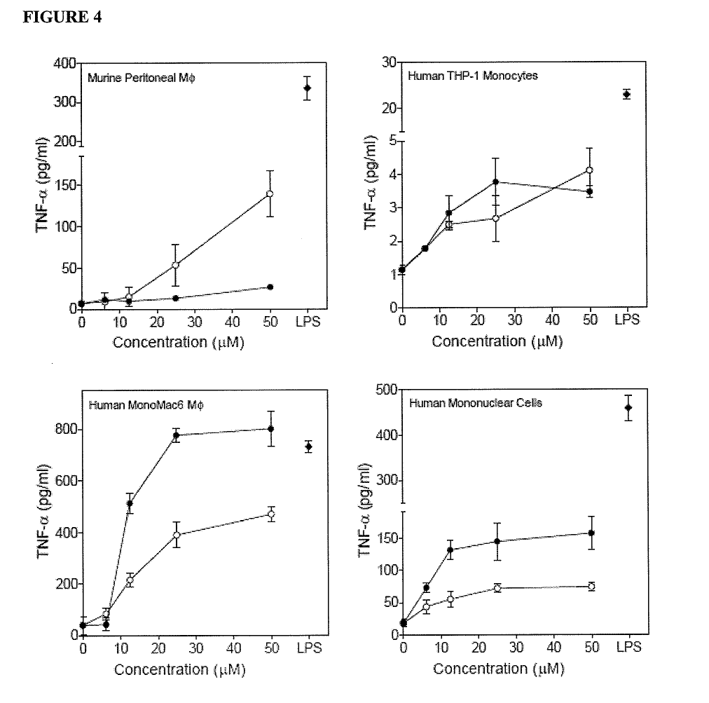

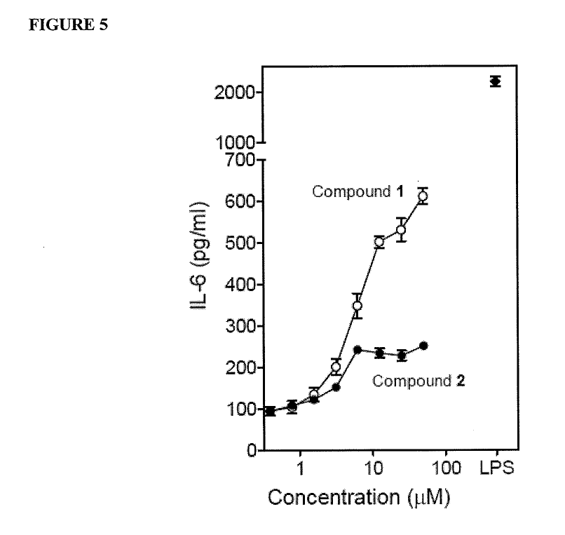

To investigate the effect of Compounds 1 and 2 on TNF-α production in macrophages of different lines and species, murine peritoneal macrophages, human monocyte/macrophage cells (THP-1 and MonoMac6), and human monocyte-derived macrophages were stimulated with various concentrations of these compounds. Both compounds induced TNF-α production in a concentration-dependent manner in murine, as well as in human cells (FIG. 4). Note, however, that there was a wide range in the level of TNF-α produced by the various cell lines. Furthermore, Compound 2 generally had much higher activity in human cells; whereas, Compound 1 was more potent in activating murine cells. To determine whether the most potent TNF-α inducers could also induce interleukin 6 (IL-6) production, we evaluated levels of IL-6 produced by J774.A1 cells treated with Compounds 1 and 2. As shown in FIG. 5, both compounds induced secretion of IL-6, with Compound 1 being the most potent of the two. In comparison, Compounds 3-5 and AG-14 all failed to induce macrophage IL-6 production (data not shown).

### Example 3

### Effect of TNF-α Inducers on Phagocyte Chemotaxis and ROS Production

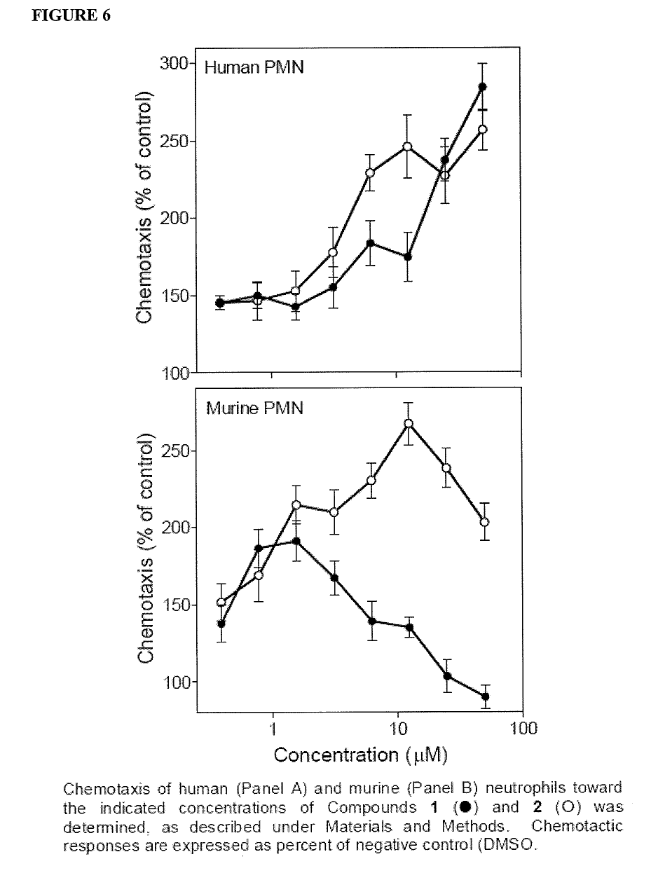

Previously, the inventors found that treatment of phagocytes with two arylcarboxylic acid hydrazides (AG-14 and AG-104) stimulated phagocyte chemotaxis (Schepetkin et al., 2007). Thus, the inventors evaluated the effects of Compounds 1 and 2 on this response in murine and human phagocytes. Both of these compounds were found to be neutrophil chemoattractants and dose-dependently induced human as well as murine neutrophil migration (FIG. 6), although the magnitude of these responses was generally lower than those induced by IL-8 or KC for human and murine neutrophils, respectively (Table 3). In comparison, maximal chemotaxis toward Compound AG-14 was equal to or much higher than the response induced by IL-8 or KC, respectively. Note that the dose-response curves for murine neutrophil chemotaxis were bellshaped; whereas, the chemotactic responses of human neutrophils increased with increasing dose of compounds up to the maximal dose tested (FIG. 6).

TABLE 3
Effect of the most potent TNF-α inducers on Ca²⁺ mobilization, chemotaxis, and ROS
production in polymorphonuclear neutrophils (PMN) and macrophages (Mφ)

| | Ca²⁺ Mobilization | | Chemotaxis | | ROS Production | |
|---|---|---|---|---|---|---|
| | EC₅₀ (μM) | | (%)^{a} | | AC50 (μM) | |
| | Human | | Human | Murine | Human | |
| Compound | PMN | Murine Mφ | PMN | PMN | PMN | Murine Mφ |
| 1 | 0.3 ± 0.2 | 24.1 ± 4.1 | 67.8 ± 9.4 | 68.9 ± 7.8 | 0.2 ± 0.02 | 25.4 ± 4.0 |
| 2 | 0.6 ± 0.4 | 2.3 ± 0.15 | 91.3 ± 1.6 | 58.0 ± 1.4 | 0.2 ± 0.04 | 8.6 ± 2.7 |
| AG-14 | 0.63 ± 0.39 | 4.2 ± 0.8 | 98.0 ± 3.4 | 174.8 ± 9.2 | 38.6 ± 3.3 | 26.9 ± 3.9 |

^{a}Chemotactic responses are expressed as percent of response induced by 50 nM IL-8 or 50 nM KC in human and murine neutrophils, respectively.

Analysis of the ability of Compounds 1 and 2 to activate ROS production in human neutrophils and murine J774.A1 macrophages showed that these compounds dose-dependently stimulated ROS generation in both types of cells, with AC50 values in the high nM to low μM range (Table 3). Likewise, Compound AG-14 activated phagocyte ROS production, which was consistent with the inventors' previous analysis of this compound (Schepetkin et al., 2007).

### Example 4

### Effect of TNF-α Inducers on Ca²⁺ Mobilization in Neutrophils and Macrophages

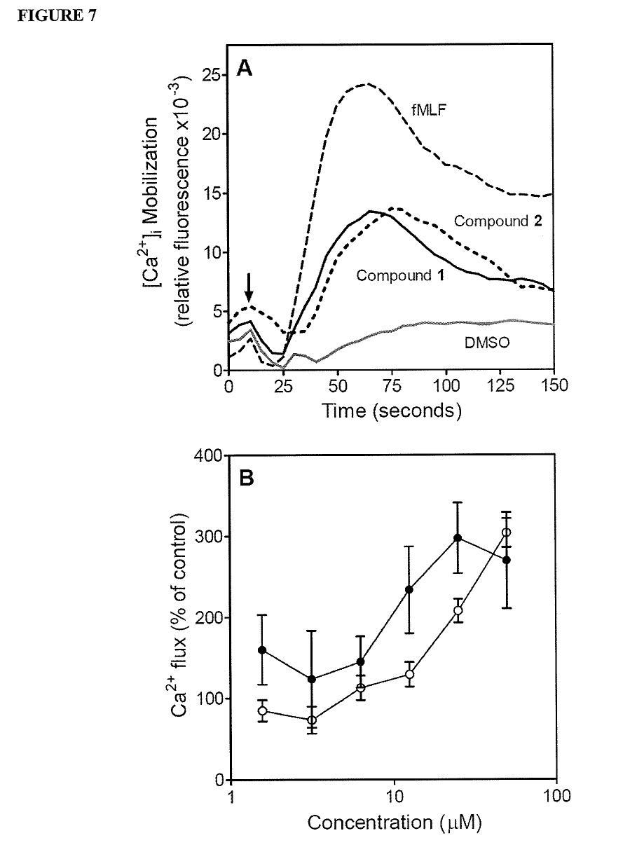

All 71 selected arylcarboxylic acid hydrazides (see Schepetkin et al., 2008) were evaluated for their ability to induce Ca²⁺ mobilization in human neutrophils. From this screening, the inventors found that 30 of the 71 compounds induced Ca²⁺ mobilization in neutrophils with EC₅₀ values ≦32 μM (Table 1), although there was not a consistent correlation between the ability to induce TNF-α production and stimulate [Ca²⁺]ᵢ flux. Nevertheless, the most potent TNF-α inducers (Table 1 and Table 2) also activated Ca²⁺ mobilization in human neutrophils. As shown in FIG. 7A, Compounds 1 and 2 induced transient increases in [Ca²⁺]ᵢ that were similar to that induced by fMLF.

Previous reports indicated that Ca²⁺ mobilization is required for TNF-α production in macrophages stimulated by different agents (Watanabe et al., 1996; Pollaud-Chemon et al., 1998; Mayne et al., 2000). Thus, we examined the effect of Compounds 1 and 2 on [Ca²⁺]ᵢ mobilization in J774.A1 macrophages. As shown in FIG. 7B, treatment of J774.A1 macrophages with Compounds 1 and 2 dose-dependently increased [Ca²⁺]i, and calculated EC₅₀ values for these compounds, as well as Compound AG-14, are shown in Table 3. By comparison, fMLF was much less potent (EC₅₀>30 μM). As a positive control, the Ca²⁺ ionophore ionomycin increased [Ca²⁺]i with in these cells (EC₅₀=0.58±0.3 μM). A substantial Ca²⁺ response to fMLF could not be obtained due to the limited solubility of fMLF (He et al., 2000); however, this result is also consistent with previous studies showing mouse cells only express low affinity receptors for fMLF (Gao et al., 1998).

### Example 5

### TNF-α Inducers Activate Ca²⁺ Mobilization Through FPR or FPRL1

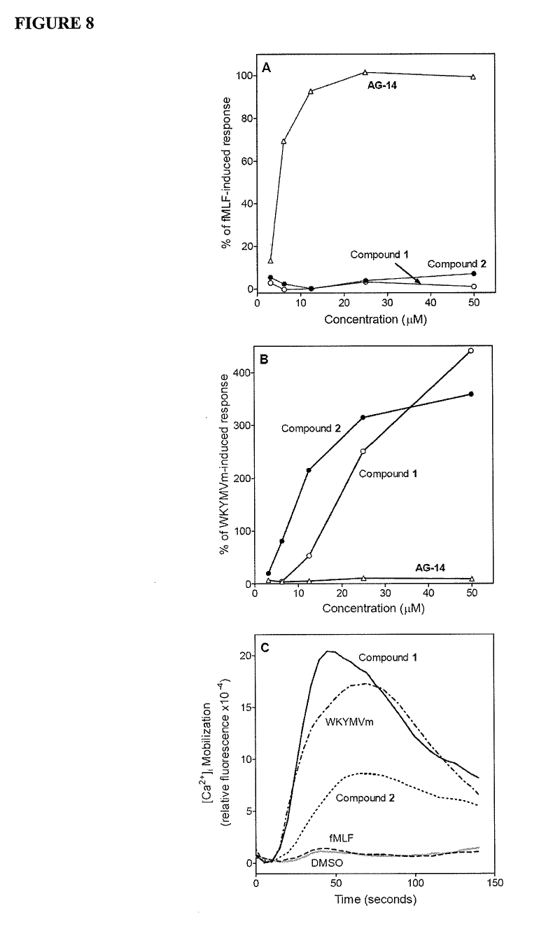

Previously, the inventors demonstrated that Compound AG-14 activated neutrophils through stimulation of FPR/FPRL1, although the receptor subtype specificity was not evaluated (Schepetkin et al., 2007). Thus, the inventors analyzed whether Compounds 1 and 2 were also FPR/FPRL1 agonists. In addition, the inventors evaluated receptor subtype specificity for these two compounds, as well as for Compound AG-14, using RBL-2H3 cells transfected with human FPR (RBL-FPR cells) or FPRL1 (RBL-FPRL1 cells) (Nanamori et al., 2004). Both Compounds 1 and 2 induced a dose-dependent increase in [Ca²⁺]ᵢ in RBL-FPRL1 cells (FIG. 7A, B and Table 4); whereas, no response was observed in wild-type or RBL-FPR cells treated with either of these compounds (FIG. 8A). In comparison, Compound AG-14 induced a dose-dependent increase in [Ca²⁺]ᵢ in RBL-FPR cells but had no effect on wild-type or RBL-FPRL1 cells (FIG. 8C and Table 4). Thus, these data indicate Compounds 1 and 2 are novel FPRL1-specific agonists, while Compound AG-14 is specific for FPR.

TABLE 4
Effect of selected compounds on Ca²⁺ mobilization in RBL-2H3 cells
transfected with human FPR (RBL-FPR cells) or FPRL1
(RBL-FPRL1 cells)

| | Ca²⁺ Mobilization EC₅₀ (μM) | | |
|---|---|---|---|
| Compound | RBL-FPR | RBL-FPRL1 | WT-RBL^{b} |
| 1 | N.A.^{a} | 19.1 | N.A. |
| 2 | N.A. | 18.3 | N.A. |
| AG-14 | 6.6 | N.A. | N.A. |

^{a}N.A., not active. No response was observed during first the 3 min after addition of compounds under investigation.
^{b}WT, wild-type non-trasfected RBL-2H3 cells.

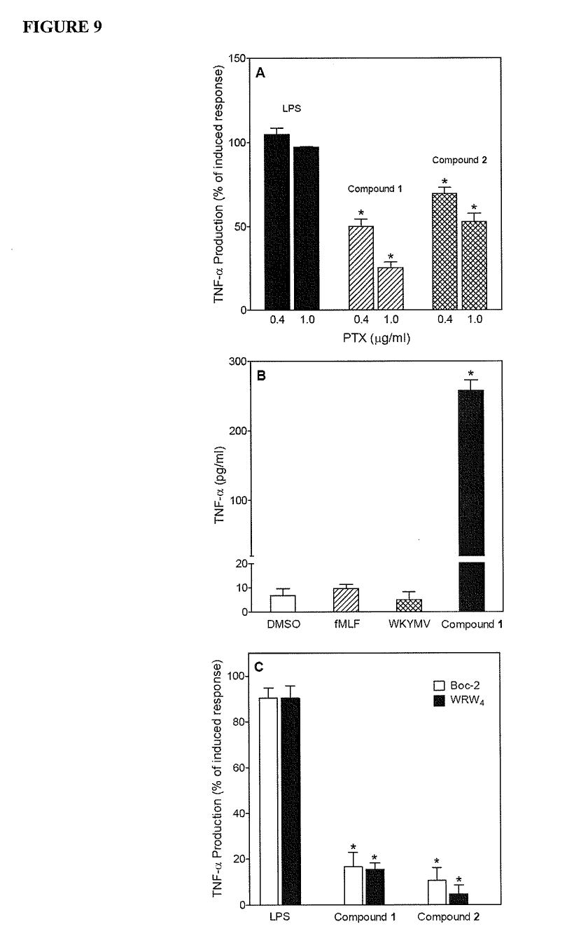

FPRL1 is a G-protein coupled receptor (GPCR) and signals through pertussis toxin (PTX)-sensitive heterotrimeric G-proteins (Gi/Go) (Migeotte et al., 2006). Thus, the inventors evaluated the effects of PTX on TNF-α production induced by Compounds 1 and 2. As shown in FIG. 9A, PTX significantly inhibited the production of TNF-α induced by both Compounds 1 and 2. In comparison, PTX had little or no effect on the LPS-induced TNF-α response. This observation is consistent with previous studies showing PTX failed to block LPS-induced production of TNF-α or other cytokines by macrophages (Zhang and Morrison, 1993; Hu et al., 2001; Katakami et al., 1988). Importantly, cell viability was not significantly affected in cells treated with PTX (data not shown). Thus, these results provide further evidence that Compounds 1 and 2 are FPRL1 agonists.

The ability to induce TNF-α production via the FPR or FPRL1 pathways was quite specific to the compounds we identified here, as both of the peptide agonists, fMLF and WKYMVm, failed to induce macrophage TNF-α production (FIG. 9B). However, these results did not rule out the possibility that Compounds 1 and 2 could be activating TNF-α production through a different pathway on these cells. To address this issue, the inventors evaluated the effects of Boc-2, an antagonist of FPR/FPRL1 (Gavins et al., 2003), and WRW4, an antagonist of FPRL1 (Bae et al., 2004) on this response. As shown in FIG. 9C, both antagonists inhibited TNF-α production induced by Compound 1 and Compound 2 in J774.A1 macrophages, directly demonstrating that both compounds were inducing TNF-α production through FPRL1.

### Example 6

### Molecular Modeling

Previously, Edwards et al. (Edwards et al., 2005) developed a pharmacophore model for FPR ligands that was based on docking of known receptor agonists and antagonists onto a homology model of the receptor. This model, which contains two acceptors for H-bonding and one hydrophobic point, was used previously to demonstrate the low-energy conformations of six small-molecule neutrophil agonists, including AG-14, fit structural requirements for ligand bioactivity (Schepetkin et al., 2007). Visual inspection of structures of Compounds 1 and 2 showed that each of these compounds contained several potential acceptors for H-bonding and at least one hydrophobic center. Thus, the inventors evaluated whether Compounds 1 and 2 also fit with this pharmacophore model.

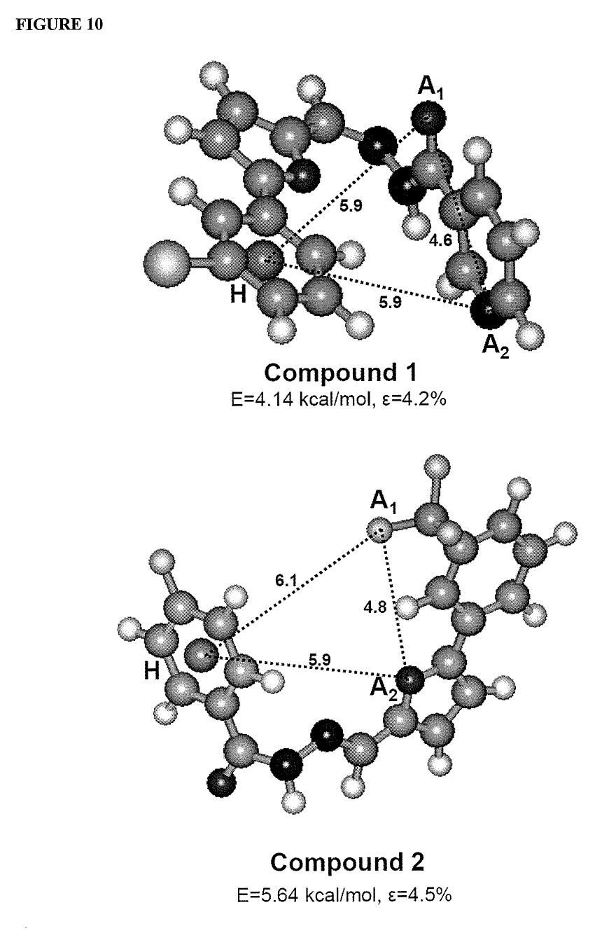

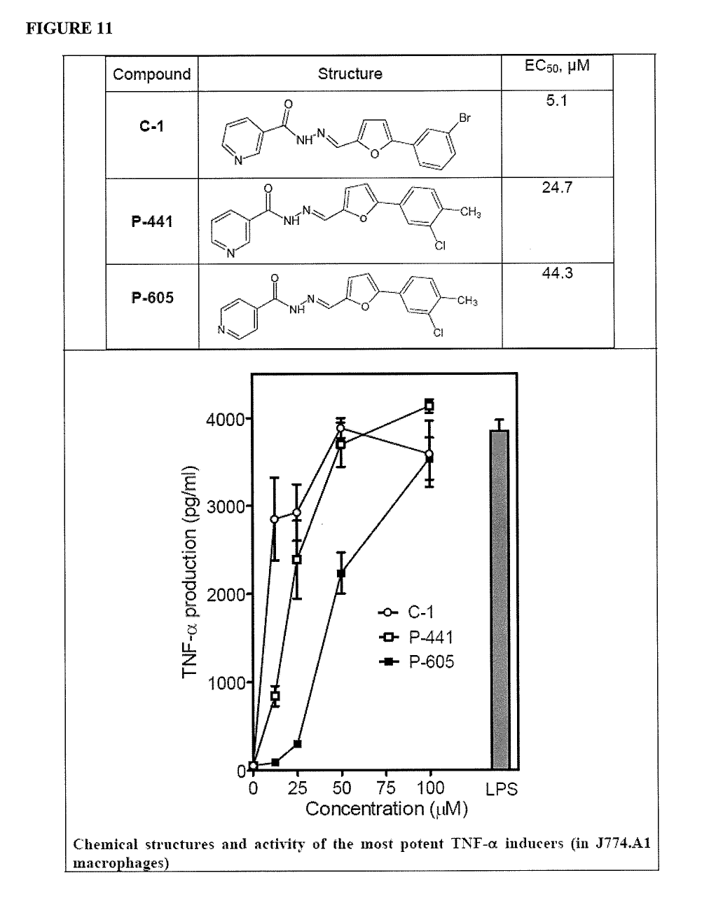

Considering that these compounds are flexible molecules, the inventors explored their potential energy surfaces using a conformational search with an MM+ force field, and the conformations within an energy gap of 6 kcal/mol over the global minimum (Nicklaus et al., 1995) were stored. The numbers of nonequivalent conformations within 6 kcal/mol of the global minimum found for the Compounds 1 and 2 were 90 and 120, respectively. Best-fit conformations for these compounds, together with calculated distances, best root-mean-square (RMS) fit (ε), and conformational energy are shown in FIG. 10. The most active compound (Compound 1) had two conformations that fit quite well into the pharmacophore model. Distances A1-H and A2-H in the conformation with the lowest RMS fit were essentially equivalent (FIG. 10), hence an additional good fit (ε=4.3%) is possible for this same conformation by switching the two H-bond acceptors such that A1 is now the pyridine nitrogen and A2 is the carbonyl oxygen. The next fit of lower quality (ε=9.1%) corresponded to a conformation with the same location of A2 and H as shown in FIG. 10, but with A1 located on the double-bonded hydrazide nitrogen. The best fit for Compound 2 (ε=4.5%) also was well within the tolerance range reported for the ligand pharmacophore (Edwards et al., 2005) (FIG. 11).

### Example 7

### Identification of Novel TNF-α Inducers and Selection of the Molecular Set

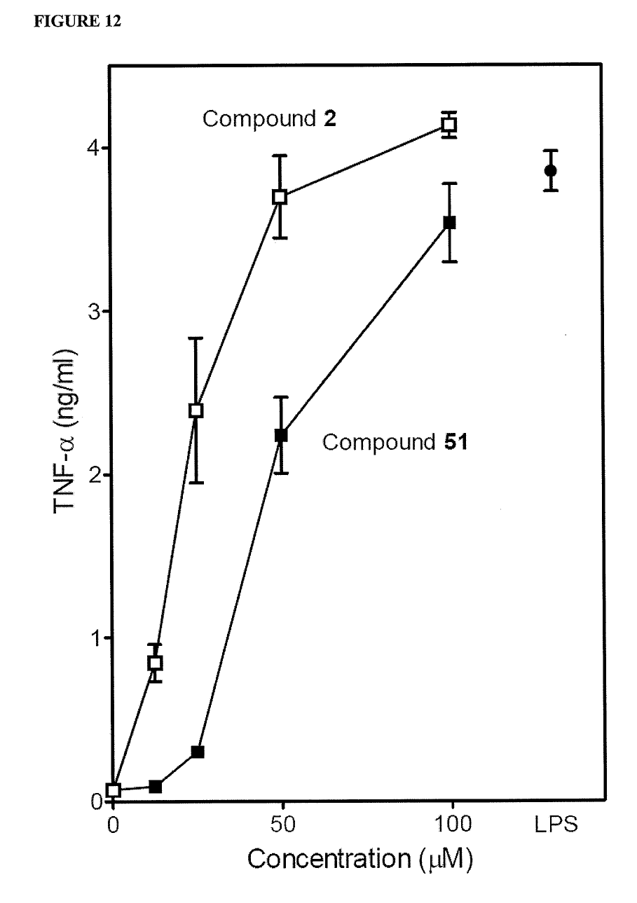

As described above, the inventors screened a series of arylcarboxylic acid hydrazide derivatives for their ability to induce macrophage tumor necrosis factor α (TNF-α) production and found that 16 compounds induced production of modest-to-high levels of TNF-α by murine and human macrophages 8. Structures of these compounds and their activity, expressed as fold-increase (FI) in macrophage TNF-α production above solvent control, were shown in Table 5 (see the Compounds in Table 5). To increase the molecular data set, the inventors selected 23 additional arylcarboxylic acid hydrazide derivatives and evaluated their ability to stimulate TNF-α production. As shown in Table 5, the inventors identified 7 additional novel compounds with varying levels of activity (Compounds 2-7, and 51 in Table 5). Derivatives of nicotinic acid (Compound 2 in Table 5) and isonicotinic acid (Compound 51 in Table 5) were the most active, inducing similar level of the TNF-α that were induced by control LPS (50 ng/ml) and the most potent of our previously identified compounds (e.g., Compound 1 in Table 5) (FIG. 12). Activation of macrophage TNF-α production was not due to endotoxin contamination, since analysis of Compounds 2 and 51 in Table 5 for endotoxin using a limulus amebocyte lysate assay showed that these compounds contained no endotoxin (below detection limit; data not shown). Furthermore, treatment with the 7 active compounds (2-7 and 51 in Table 5), as well as with the inactive compounds (11-22 and 83-86 in Table 5) from our set, had no effect on cell viability in J774.A1 macrophages, indicating lack of cytotoxicity at concentrations ≦50 μM (data not shown).

For SAR analysis, the total set of the arylcarboxylic acid hydrazide derivatives (Compounds 1-86 in Table 5) was divided into two activity classes based on their experimentally determined activity. Compounds that induced macrophage TNF-α production (FI≧2) were classified as “Active” (23 compounds); whereas, inactive derivatives were placed in the nonactive group labeled “NA” (63 compounds).

TABLE 5
Effect of arylcarboxylic acid hydrazide derivatives on macrophage TNF-α production
A. (2-furyl)methylene-hydrazides of nicotinic acid

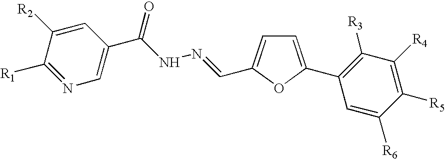

| Compound | R1 | R2 | R3 | R4 | R5 | R6 | FIa |
|---|---|---|---|---|---|---|---|
| 1 | H | H | H | Br | H | H | 50 |
| 2 | H | H | H | Cl | CH₃ | H | 60 |
| 3 | H | Br | H | H | Cl | H | 25 |
| 4 | CH₃ | H | H | Cl | CH₃ | H | 21 |
| 5 | H | H | H | Cl | H | H | 17 |
| 6 | H | H | H | H | Cl | H | 15 |
| 7 | H | Br | H | Cl | CH₃ | H | 10 |
| 8 | CH₃ | H | H | CF₃ | H | H | <5 |
| 9 | CH₃ | H | Cl | H | Cl | Cl | <5 |
| 10 | H | H | H | COOH | OH | H | N.A. |
| 11 | H | H | H | H | Br | H | N.A. |
| 12 | CH₃ | H | H | H | Cl | H | N.A. |
| 13 | CH₃ | H | H | Cl | H | H | N.A. |
| 14 | H | H | Cl | H | Cl | Cl | N.A. |
| 15 | H | Br | Cl | H | Cl | H | N.A. |
| 16 | H | H | Cl | Cl | H | H | N.A. |
| 17 | H | H | Cl | H | H | H | N.A. |
| 18 | CH₃ | H | H | Br | H | H | N.A. |
| 19 | CH₃ | H | Cl | Cl | H | H | N.A. |
| 20 | CH₃ | H | Cl | H | Cl | H | N.A. |
| 21 | CH₃ | H | Cl | H | H | Cl | N.A. |
| 22 | CH₃ | H | H | Cl | Cl | H | N.A. |

B. (2-furyl)methylene-hydrazides of benzoic acid

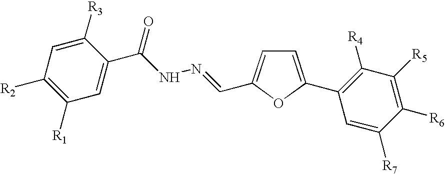

| Compound | R1 | R2 | R3 | R4 | R5 | R6 | R7 | FI |
|---|---|---|---|---|---|---|---|---|
| 23 | H | F | H | H | CF₃ | H | H | 35 |
| 24 | H | H | H | Cl | Cl | H | H | 8 |
| 25 | NO2 | H | H | H | CF₃ | H | H | <5 |
| 26 | H | Cl | Cl | Cl | H | H | H | <5 |
| 27 | H | NO2 | H | Cl | H | H | Cl | <5 |
| 28 | I | H | Cl | H | CF₃ | H | H | <5 |
| 29 | H | Br | H | H | CF₃ | H | H | N.A. |
| 30 | H | H | NO2 | Cl | H | Cl | H | N.A. |
| 31 | H | H | OH | H | Cl | Cl | H | N.A. |
| 32 | H | NO2 | H | H | Cl | H | H | N.A. |
| 33 | H | OCH₃ | H | H | COOH | Cl | H | N.A. |
| 34 | H | NO2 | H | H | Cl | OCH₃ | H | N.A. |
| 35 | OH | H | H | H | H | NO2 | H | N.A. |
| 36 | H | t-butyl | H | H | H | NO2 | H | N.A. |

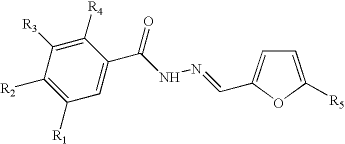

| Compound | R1 | R2 | R3 | R4 | R5 | FI |
|---|---|---|---|---|---|---|
| 37 | H | Br | H | OH | H | N.A. |
| 38 | H | CH₃ | H | H | CH₃ | N.A. |
| 39 | H | H | H | H | H | N.A. |
| 40 | H | OH | H | OH | NO2 | N.A. |
| 41 | NO2 | H | NO2 | OH | NO2 | N.A. |
| 42 | H | Cl | H | H | H | N.A. |
| 43 | Br | H | H | OCH₃ | CH₃ | N.A. |
| 44 | H | Br | H | OCH₃ | H | N.A. |
| 45 | H | 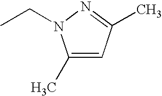 | H | H | H | N.A. |
| 46 | H | H | H | H | Br | N.A. |
| 47 | H | H | F | H | H | N.A. |
| 48 | H | 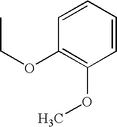 | H | H | H | N.A. |
| 49 | H | Cl | H | Cl | H | N.A. |
| 50 | H | H | H | F | I | N.A. |

C. Other derivatives

| Compound | Structure | FI |
|---|---|---|
| 51 | 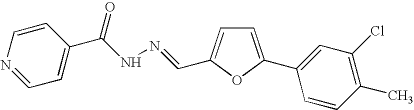 | 50 |
| 52 | 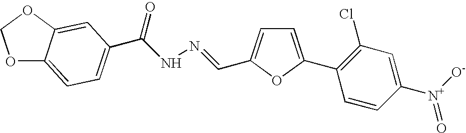 | 35 |
| 53 | 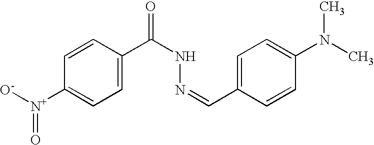 | 25 |
| 54 | 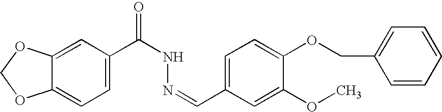 | 7 |
| 55 | 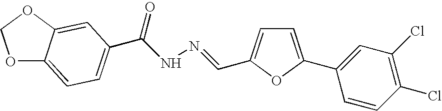 | <5 |
| 56 | 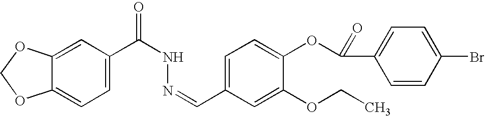 | <5 |
| 57 | 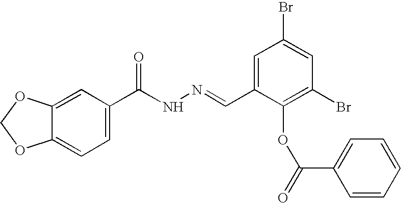 | <5 |
| 58 | 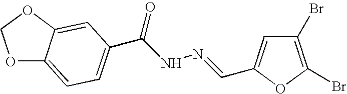 | <5 |
| 59 | 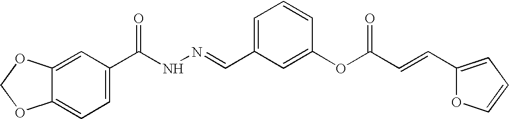 | N.A. |
| 60 | 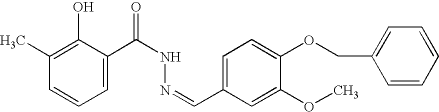 | N.A. |
| 61 | 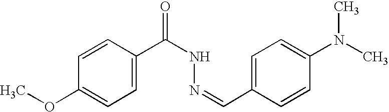 | N.A. |
| 62 | 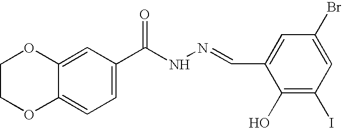 | N.A. |
| 63 | 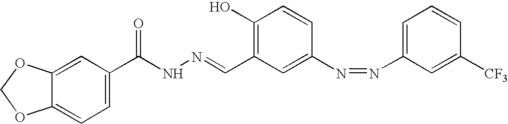 | N.A. |
| 64 | 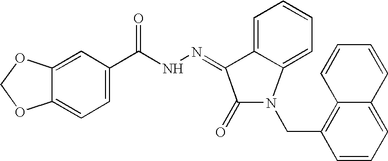 | N.A. |
| 65 | 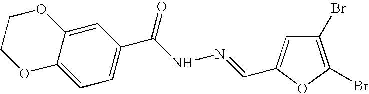 | N.A. |
| 66 | 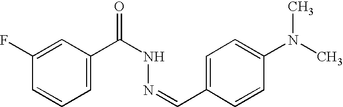 | N.A. |
| 67 | 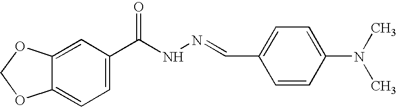 | N.A. |
| 68 | 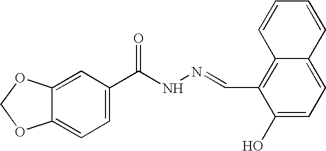 | N.A. |
| 69 | 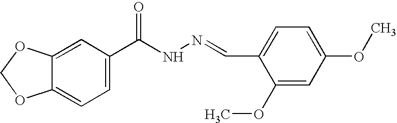 | N.A. |
| 70 | 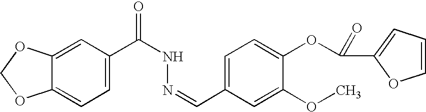 | N.A. |
| 71 | 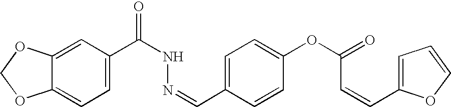 | N.A. |
| 72 | 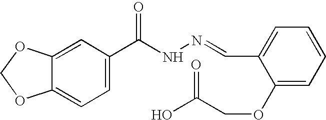 | N.A. |
| 73 | 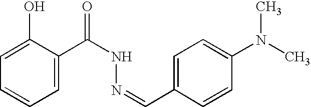 | N.A. |
| 74 | 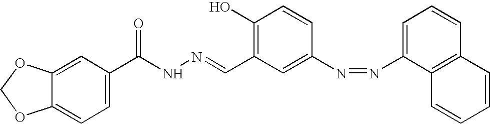 | N.A. |
| 75 | 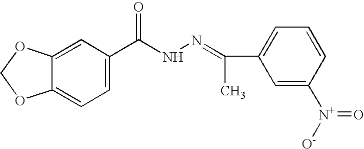 | N.A. |
| 76 | 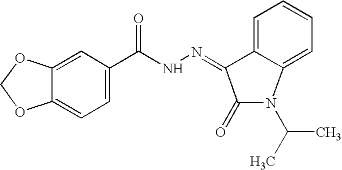 | N.A. |
| 77 | 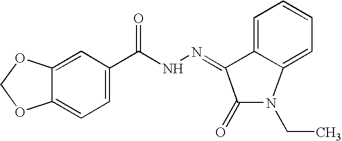 | N.A. |
| 78 | 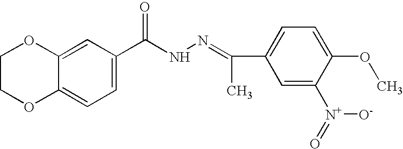 | N.A. |
| 79 | 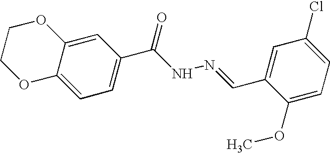 | N.A. |
| 80 | 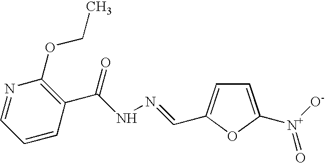 | N.A. |
| 81 | 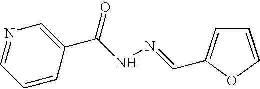 | N.A. |
| 82 | 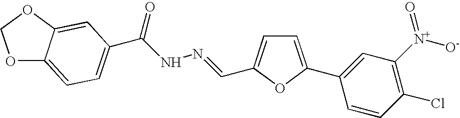 | N.A. |
| 83 | 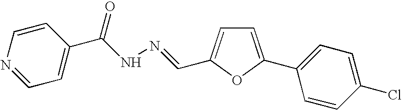 | N.A. |
| 84 | 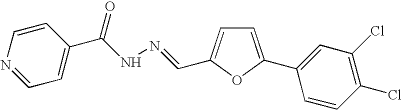 | N.A. |
| 85 | 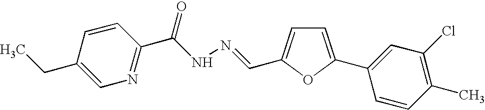 | N.A. |
| 86 | 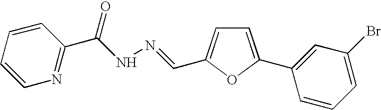 | N.A. |

^{a}Macrophage TNF-α production induced by 50 μM of the indicated compound is shown as fold-increase
(FI) above response to vehicle (DMSO) control. Activity of Compounds 2-7, 11-22, 51, and 83-86 was evaluated in the present work. Data for Compounds 1, 8-10, 23-50, and 52-82 were from above examples.

### Example 8

### Descriptors

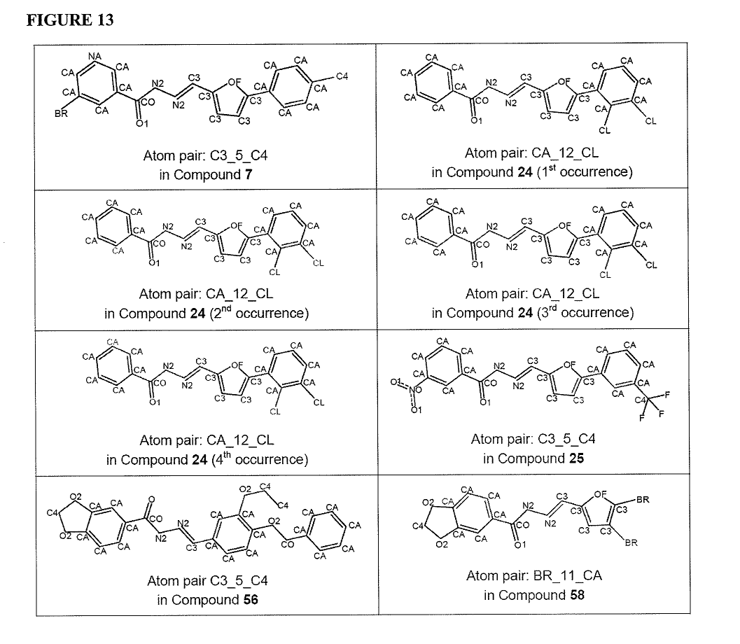

Atom pairs were automatically generated from bond connectivity of the arylcarboxylic acid hydrazides and are specified in terms of types of the two atoms in a pair separated by the number of chemical bonds in the structural formula (Carhart et al., 1985). As described previously (Khlebnikov et al., 2008), the inventors used the atom type names from MM+ force field, as implemented in HyperChem. According to this scheme, specific atom pairs are defined as T1_D_T2, where T1 and T2 are the atom types assigned by HyperChem, and D is the number of chemical bonds in the shortest path between the two atoms (see Example 1). HyperChem output in a HIN file format was entered directly into our CHAIN program, which generated all possible atom pairs and frequencies of their occurrence in each of the 86 hydrazides. These frequencies were considered as values of the corresponding atom pair descriptors. Examples of atom pairs are shown in FIG. 13. Note that atom pair descriptors are easily interpretable in terms of standard chemical formulae. Thus, BR_{—}11_CA indicates the simultaneous presence of bromine atom and an aromatic ring in the opposite sides of a molecule (see FIG. 13). It should be noted that, although atom naming was taken from MM+ force field, performing MM+ molecular mechanics optimization itself is not necessary because only bond connectivity, but not geometry, is important for the atom pair calculation.

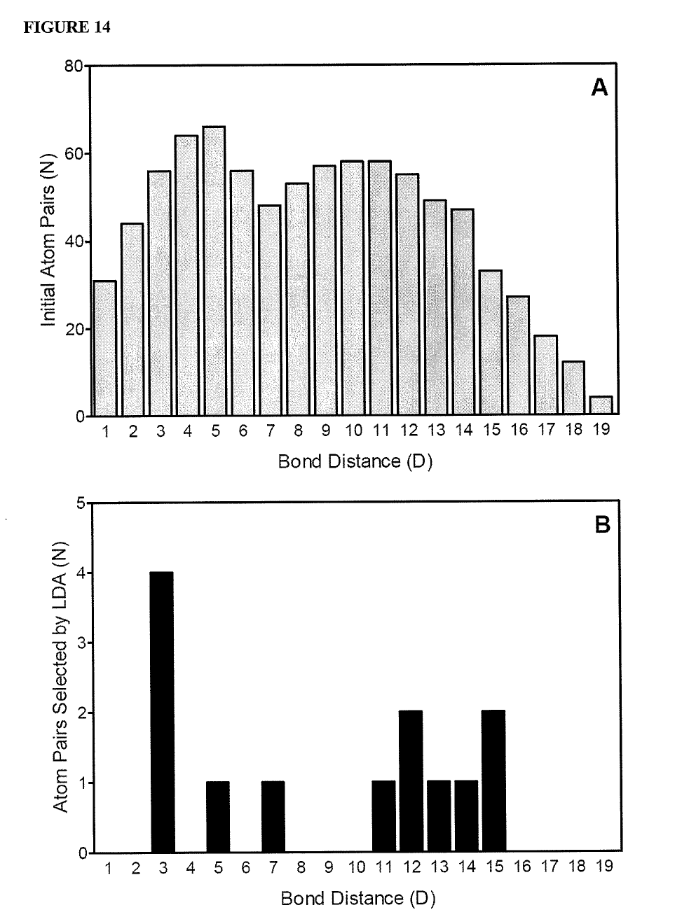

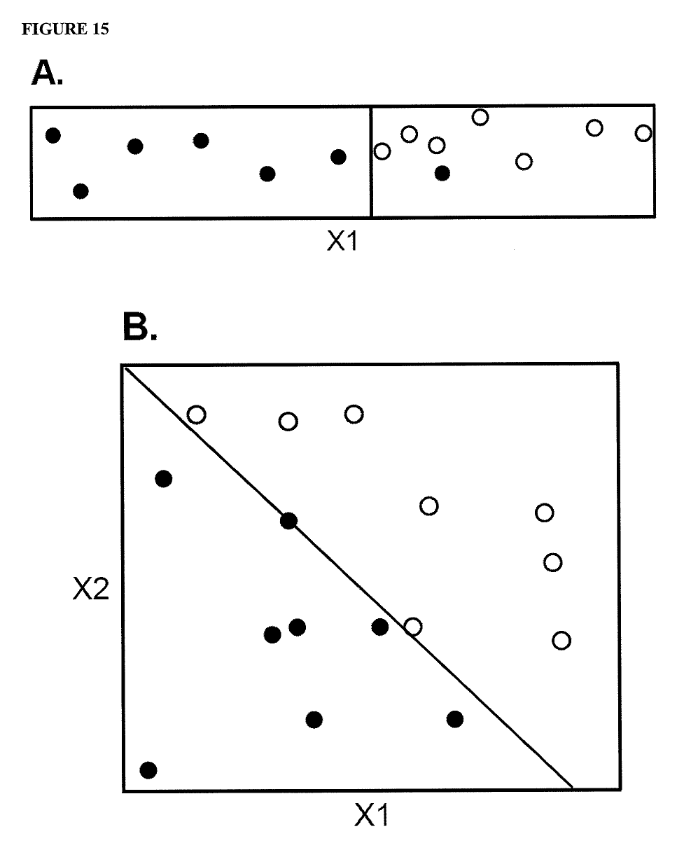

In total, 836 unique atom pairs were generated for all 86 hydrazides, and a histogram of the number of atom pairs with different bond distances is presented in FIG. 14A. Note that the histogram has two maxima at 5 and 10-11 chemical bonds, which is in agreement with the dumbbell shape of most compounds in our set. Indeed, all of the molecules contain two bulky moieties connected by the hydrazide linker. Hence, the relatively “short” atom pairs originated from the same moiety, as well as the much “longer” atom pairs representing atoms in the two different moieties prevail in the total number of 836 descriptors generated.

### Example 9

### Linear Discriminant Analysis

One of the most powerful pattern recognition techniques is linear discriminant analysis (LDA), and the inventors applied it to SAR analysis of compounds with elastase inhibitory activity (Khlebnikov et al., 2008). Likewise, the inventors used LDA here as a basic methodology for SAR classification of the 86 hydrazide derivatives. Taking into account that classical LDA is unable to handle as many as 836 descriptors for 86 compounds, the inventors performed advanced LDA with the Forward Stepwise option available in STATISTICA 6.0. At each step, descriptors were successively included or excluded until no significant (p<0.05) improvement of the model was achieved. This procedure led to the selection of only 14 significant variables from the initially generated 836 descriptors.

The following atom pairs were selected by Forward Stepwise LDA: C4_{—}2_NA, C3_{—}3_CL, C4_{—}3_O2, CO_{—}3_NA, CO_{—}3_NO, C3_{—}5_C4, C4_{—}7_OF, BR_{—}11_CA, CA_{—}12_CL, CL_{—}12_NA, BR_{—}13_C4, C4_{—}14_NO, BR_{—}15_C4, and CL_{—}15_O2. Use of the classification functions obtained with these pairs resulted in 95.3% correct classification: 20 of 23 active and 62 of 63 inactive hydrazides were correctly classified to their experimentally-determined activity. In addition, values of the 14 atom pairs selected were not mutually correlated with each other (r≦0.7), i.e. they can be regarded as independent variables.

In order to further decrease the number of descriptors, the inventors performed LDA analysis with the Best Subset Search option, starting from 14 atom pairs selected after the first run of the LDA procedure and found that the best subset consisted of 13 atom pairs as listed above, but with C4_{—}2_NA excluded. These variables provided the least misclassification error among all other possible subsets of different sizes chosen from 14 descriptors, and the SAR model obtained had an improved quality of classification: 96.5% compounds were classified correctly compared to their experimental activity (Tables 6 and Table 7). This LDA model can be presented by two classification functions F(Active) and F(NA):

F(Active)=−11.81+15.52C3_{—}3_CL−16.49C4_{—}3_O2+5.62CO_{—}3_NA−19.42CO_{—}3_NO+19.08C3_{—}5_C4−9.11C4_{—}7_OF+10.82BR_{—}11_CA+2.17CA_{—}12_CL−22.61CL_{—}12_NA−9.26BR_{—}13_C4+10.96C4_{—}14_NO−15.18BR_{—}15_C4+7.34CL_{—}15_O2 (Eq. 1)

F(NA)=−0.725+0.567C3_{—}3_CL+0.514C4_{—}3_O2+1.213CO_{—}3_NA+0.160CO_{—}3_NO+1.648C3_{—}5_C4+0.859C4_{—}7_OF+1.237BR_{—}11_CA+0.478CA_{—}12_CL−0.915CL_{—}12_NA+0.060BR_{—}13_C4+1.229C4_{—}14_NO−0.744BR_{—}15_C4+0.802CL_{—}15_O2 (Eq. 2)

TABLE 6
Classification matrices for linear discriminant analysis (LDA) and classification
tree analysis with linear combination splits (CTLCS)

| | Calculated Classification | | | | | | | | |
|---|---|---|---|---|---|---|---|---|---|
| | | | | | | | Classification | | |
| | | | | | | | Tree with | | |
| Experimentally | LDA model | | | CTLCS model | | | Univariate Splits | | |
| Determined | | | Accuracy | | | Accuracy | | | Accuracy |
| Classification | Active | NA | (%) | Active | NA | (%) | Active | NA | (%) |
| Active | 20 | 3 | 87.0 | 20 | 3 | 87.0 | 16 | 7 | 69.6 |
| NA | 0 | 63 | 100.0 | 4 | 59 | 93.7 | 6 | 57 | 90.5 |
| Total | 20 | 66 | 96.5 | 24 | 62 | 91.9 | 22 | 64 | 84.9 |

The number of compounds correctly classified by the model is indicated in bold.

TABLE 7
Experimentally determined, SAR-calculated, and LOO-predicted classes of
macrophage TNF-α inducing activity for all 86 arylcarboxylic acid hydrazide derivatives

| | | | | | | Classification Tree with Univariate Splits | | | |
|---|---|---|---|---|---|---|---|---|---|
| | | | | | | Atom Pairs and Frequency | | | |
| | | LDA Model | | CTLCS Model | | of Occurrence | | | |
| Compound | Determined | Calculated | LOO-predicted | Calculated | LOO-predicted | C3_5_C4 | CA_12_CL | BR_11_CA | Calculated^{a} |
| 1 | Active | Active | Active | Active | Active | 0 | 0 | 1→ | NA |
| 2 | Active | Active | Active | Active | Active | 1 | 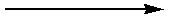 | | Active |
| 3 | Active | Active | Active | Active | Active | 0 | 1 | 1→ | NA |
| 4 | Active | Active | Active | Active | Active | 1 | 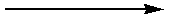 | | Active |
| 5 | Active | NA | NA | NA | NA | 0 | 2 | 0→ | NA |
| 6 | Active | NA | NA | NA | NA | 0 | 1 | 0→ | NA |
| 7 | Active | Active | Active | Active | Active | 1 | 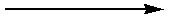 | | Active |
| 8 | Active | Active | Active | Active | Active | 1 | 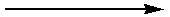 | | Active |
| 9 | Active | NA | NA | NA | NA | 0 | 4 | 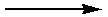 | Active |
| 10 | NA | NA | NA | NA | NA | 0 | 0 | 0→ | NA |
| 11 | NA | NA | NA | NA | NA | 0 | 0 | 0→ | NA |
| 12 | NA | NA | NA | NA | NA | 0 | 1 | 0→ | NA |
| 13 | NA | NA | NA | NA | NA | 0 | 2 | 0→ | NA |
| 14 | NA | NA | NA | NA | NA | 0 | 4 | 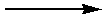 | Active |
| 15 | NA | NA | Active | Active | Active | 0 | 2 | 1→ | NA |
| 16 | NA | NA | NA | NA | NA | 0 | 3 | 0→ | NA |
| 17 | NA | NA | NA | NA | NA | 0 | 1 | 0→ | NA |
| 18 | NA | NA | NA | NA | NA | 0 | 0 | 1→ | NA |
| 19 | NA | NA | NA | NA | NA | 0 | 3 | 0→ | NA |
| 20 | NA | NA | NA | NA | NA | 0 | 2 | 0→ | NA |
| 21 | NA | NA | NA | NA | NA | 0 | 3 | 0→ | NA |
| 22 | NA | NA | NA | Active | Active | 0 | 3 | 0→ | NA |
| 23 | Active | Active | Active | Active | Active | 1 | 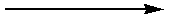 | | Active |
| 24 | Active | Active | Active | Active | Active | 0 | 4 | 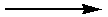 | Active |
| 25 | Active | Active | Active | Active | Active | 1 | 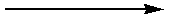 | | Active |
| 26 | Active | Active | Active | Active | Active | 0 | 5 | 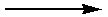 | Active |
| 27 | Active | Active | Active | Active | Active | 0 | 4 | 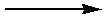 | Active |
| 28 | Active | Active | Active | Active | Active | 1 | 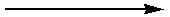 | | Active |
| 29 | NA | NA | NA | NA | NA | 1 | 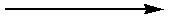 | | Active |
| 30 | NA | NA | Active | NA | Active | 0 | 3 | 0→ | NA |
| 31 | NA | NA | NA | NA | NA | 0 | 3 | 0→ | NA |
| 32 | NA | NA | NA | NA | NA | 0 | 2 | 0→ | NA |
| 33 | NA | NA | NA | NA | NA | 0 | 1 | 0→ | NA |
| 34 | NA | NA | NA | NA | NA | 0 | 2 | 0→ | NA |
| 35 | NA | NA | NA | NA | NA | 0 | 0 | 0→ | NA |
| 36 | NA | NA | NA | NA | NA | 0 | 0 | 0→ | NA |
| 37 | NA | NA | NA | NA | NA | 0 | 0 | 0→ | NA |
| 38 | NA | NA | NA | NA | NA | 0 | 0 | 0→ | NA |
| 39 | NA | NA | NA | NA | NA | 0 | 0 | 0→ | NA |
| 40 | NA | NA | NA | NA | NA | 0 | 0 | 0→ | NA |
| 41 | NA | NA | NA | NA | NA | 0 | 0 | 0→ | NA |
| 42 | NA | NA | NA | NA | NA | 0 | 0 | 0→ | NA |
| 43 | NA | NA | NA | NA | NA | 0 | 0 | 0→ | NA |
| 44 | NA | NA | NA | NA | NA | 0 | 0 | 0→ | NA |
| 45 | NA | NA | NA | NA | NA | 0 | 0 | 0→ | NA |
| 46 | NA | NA | NA | Active | Active | 0 | 0 | 1→ | NA |
| 47 | NA | NA | NA | NA | NA | 0 | 0 | 0→ | NA |
| 48 | NA | NA | NA | NA | NA | 0 | 0 | 0→ | NA |
| 49 | NA | NA | NA | NA | NA | 0 | 0 | 0→ | NA |
| 50 | NA | NA | NA | NA | NA | 0 | 0 | 0→ | NA |
| 51 | Active | Active | Active | Active | Active | 1 | 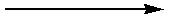 | | Active |
| 52 | Active | Active | Active | Active | Active | 0 | 2 | 0→ | NA |
| 53 | Active | Active | NA | Active | NA | 0 | 0 | 0→ | NA |
| 54 | Active | Active | Active | Active | Active | 1 | 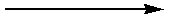 | | Active |
| 55 | Active | Active | NA | Active | NA | 0 | 3 | 0→ | NA |
| 56 | Active | Active | Active | Active | Active | 1 | 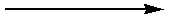 | | Active |
| 57 | Active | Active | Active | Active | Active | 0 | 0 | 2→ | Active |
| 58 | Active | Active | Active | Active | Active | 0 | 0 | 2→ | Active |
| 59 | NA | NA | NA | NA | NA | 0 | 0 | 0→ | NA |
| 60 | NA | NA | Active | NA | Active | 1 | 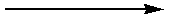 | | Active |
| 61 | NA | NA | NA | NA | NA | 0 | 0 | 0→ | NA |
| 62 | NA | NA | NA | NA | NA | 0 | 0 | 1→ | NA |
| 63 | NA | NA | NA | NA | NA | 0 | 0 | 0→ | NA |
| 64 | NA | NA | NA | NA | NA | 0 | 0 | 0→ | NA |
| 65 | NA | NA | NA | NA | NA | 0 | 0 | 2→ | Active |
| 66 | NA | NA | NA | NA | NA | 0 | 0 | 0→ | NA |
| 67 | NA | NA | NA | NA | NA | 0 | 0 | 0→ | NA |
| 68 | NA | NA | NA | NA | NA | 0 | 0 | 0→ | NA |
| 69 | NA | NA | NA | NA | NA | 0 | 0 | 0→ | NA |
| 70 | NA | NA | Active | NA | Active | 1 | 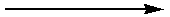 | | Active |
| 71 | NA | NA | NA | NA | NA | 0 | 0 | 0→ | NA |
| 72 | NA | NA | NA | NA | NA | 0 | 0 | 0→ | NA |
| 73 | NA | NA | NA | NA | NA | 0 | 0 | 0→ | NA |
| 74 | NA | NA | NA | NA | NA | 0 | 0 | 0→ | NA |
| 75 | NA | NA | NA | NA | NA | 0 | 0 | 0→ | NA |
| 76 | NA | NA | NA | NA | NA | 0 | 0 | 0→ | NA |
| 77 | NA | NA | NA | NA | NA | 0 | 0 | 0→ | NA |
| 78 | NA | NA | NA | NA | NA | 0 | 0 | 0→ | NA |
| 79 | NA | NA | NA | NA | NA | 0 | 0 | 0→ | NA |
| 80 | NA | NA | NA | NA | NA | 0 | 0 | 0→ | NA |
| 81 | NA | NA | NA | NA | NA | 0 | 0 | 0→ | NA |
| 82 | NA | NA | NA | NA | Active | 0 | 1 | 0→ | NA |
| 83 | NA | NA | NA | NA | NA | 0 | 1 | 0→ | NA |
| 84 | NA | NA | NA | NA | NA | 0 | 3 | 0→ | NA |
| 85 | NA | NA | NA | NA | NA | 1 | 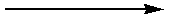 | | Active |
| 86 | NA | NA | NA | Active | Active | 0 | 0 | 1→ | NA |

According to these equations, a compound will be classified as “Active” if the value of F(Active)>F(NA), and vice versa. The classifications observed and calculated by the LDA model for Compounds 1-86 in Table 5 are shown in Table 7.

The predictive ability of the LDA model was evaluated by the leave-one-out (LOO) procedure. The LOO prediction resulted in 89.5% correct classification, and 18 of 23 active and 59 of 63 inactive hydrazides were correctly predicted for their TNF-α induction activity classes (Table 7). Thus, these results confirm usefulness of the LDA model for a priori evaluation of macrophage TNF-α inducing activity of arylcarboxylic acid hydrazides.

Although 13 atom pair descriptors were utilized in the derived LDA model, this number should not be regarded as too large. Conventionally, the recommended number of variables for SAR and QSAR models, from a statistical point of view, should be ≦20% of the number of compounds. Hence, the number of atom pairs selected is reasonable for 86 hydrazide derivatives investigated. Additionally, all coefficients of the classification functions (Eq. 1 and 2) were significant according to the Fisher criterion.

The atom pairs involved in Eq. 1 and 2 are not uniformly distributed in number of chemical bonds D. FIG. 14B shows that six atom pairs used in the LDA model have bond distances from 3 to 7, while the other seven descriptors are characterized by D values from 11 to 15. Indeed, this distribution is a reflection of total atom pair distribution (FIG. 14A), which is conditioned by the dumbbell shape of the compounds investigated. On the other hand, the importance of “longer” atom pairs for SAR classification supports the supposition that a biological target interacts with the entire hydrazide molecule, rather than with metabolites of a smaller size.

### Example 10

### Classification Tree Analysis with Linear Combination Splits

In the SAR analysis of N-benzoylpyrazoles with elastase inhibitory activity, the inventors also used LDA methodology (Khlebnikov et al.); however, its use was preceded by application of one-way analysis of variance (ANOVA) (Lindman et al., 1974) for preliminary selection of descriptors having significant differences between in-class and total variances. This led to a substantial decrease in the number of atom pairs to reduce dimensionality of the data matrix for further SAR analysis. Since each descriptor selected by ANOVA has one-dimensional separation of classes, compounds from different groups are characterized by relatively distinct areas of data point projections on a single coordinate axis associated with a given descriptor (e.g., see FIG. 15A).

It should be noted that in the case of hydrazides 1-86 in Table 5, the pre-selection of atom pairs by ANOVA did not result in a satisfactory SAR model for predicting their macrophage TNF-α inducing activity if the LDA method was applied to the ANOVA-selected descriptors. Instead, good classification was achieved by stepwise LDA applied to the initial non-reduced data matrix, as described above. Notably, only three atom pairs (C3_{—}5_C4, CA_{—}12_CL, and C4_{—}14_NO) of thirteen descriptors involved in Eq. 1 and 2 were selected in the trial run of ANOVA and thus had approximately one-dimensional class separation, as exemplified in FIG. 15A. The other 10 atom pairs had occurrences that non-significantly differed between classes of active and nonactive compounds. These atom pair descriptors clearly belong to another type where the activity classes were separated in higher-dimensional subspaces of such descriptors (see two-dimensional example in FIG. 15B). Although the projections of data points for both classes in this example are approximately uniformly distributed on each coordinate axis, there exists a line of good separation, and such descriptors appear to be very useful for SAR analysis, as demonstrated above by LDA. In a more common case of higher dimensionality, there may exist a hyper-plane separating two classes of compounds. Taking into account the distribution character of data points for Compounds 1-86 of Table 5 in descriptor space, we attempted to apply a methodology known as classification tree analysis with linear combination splits (CTLCS) (Loh et al., 1997).

In this approach, a logical tree was created where a split condition for each tree node depends on a linear combination of several descriptors. The inventor found that the best classification tree for Compounds 1-86 of Table 5 had just one split. The 13 atom pairs utilized in the LDA model (see Eq. 1 and 2) were used as a basis in the CTLCS approach, and all pairs were included in the function F(x) (Eq. 3), indicating again that all 13 descriptors were important for prediction of the correct biological activity class.

F(x)=−0.122+0.192C3_{—}3_CL−0.219C4_{—}3_O2+0.057CO_{—}3_NA−0.252CO_{—}3_NO+0.224C3_{—}5_C4−0.128C4_{—}7_OF+0.123BR_{—}11_CA+0.022CA_{—}12_CL−0.279CL_{—}12_NA−0.120BR_{—}13_C4+0.125C4_{—}14_NO−0.186BR_{—}15_C4+0.084CL_{—}15_O2 (Eq. 3)

According to the split condition, a compound would be classified as inactive if F(x)≦0; otherwise a compound belongs to the “Active” class. The classification matrix obtained by the CTLCS method is shown in Table 6. The activity classes were predicted correctly for 20 of 23 active and 59 of 63 inactive hydrazides, resulting in a total accuracy of fitting 91.9%. The calculated and LOO-predicted classes for individual compounds are shown in Table 7. In 73 of 86 cases (84.9%), a priori prediction of activity class by the LOO procedure was correct. While LDA classification by Eq. 1 and 2 had better characteristics of fitting and prediction (Table 6), the CTLCS model was two-fold simpler in the amount of calculation necessary for a compound classification. Satisfactory results obtained by the one-split tree based on linear combination of variables indicates that the descriptor space in divided into two areas by a hyper-plane expressed by Eq. 3. Each of these areas preferentially contains data points for compounds of a single activity class, such as in the two-dimensional example given in FIG. 15B. Such well-organized data in a space of atom pair descriptors demonstrates a powerful ability of atom pairs to separate compounds of different activity in SAR analysis.

It should be noted that most of the incorrect classifications by both the LDA and CTLCS methods were made in the subset of nicotinic acid hydrazide derivatives 1-22 (Table 7). Hence, some structural or physico-chemical peculiarities of nicotinic acid hydrazides may be reflected non-significantly in the entire matrix of atom pair descriptors.

### Example 11

### Classification Tree Analysis with Univariate Splits

Although the LDA and CTLCS models had high fitting and predictive abilities, it is difficult to formulate these models in a set of intuitively understandable “chemical” rules. The methodology of binary classification tree analysis with univariate splits 18 is more suitable for deriving simplified SAR rules, while being less complex than the LDA or CTLCS methods.

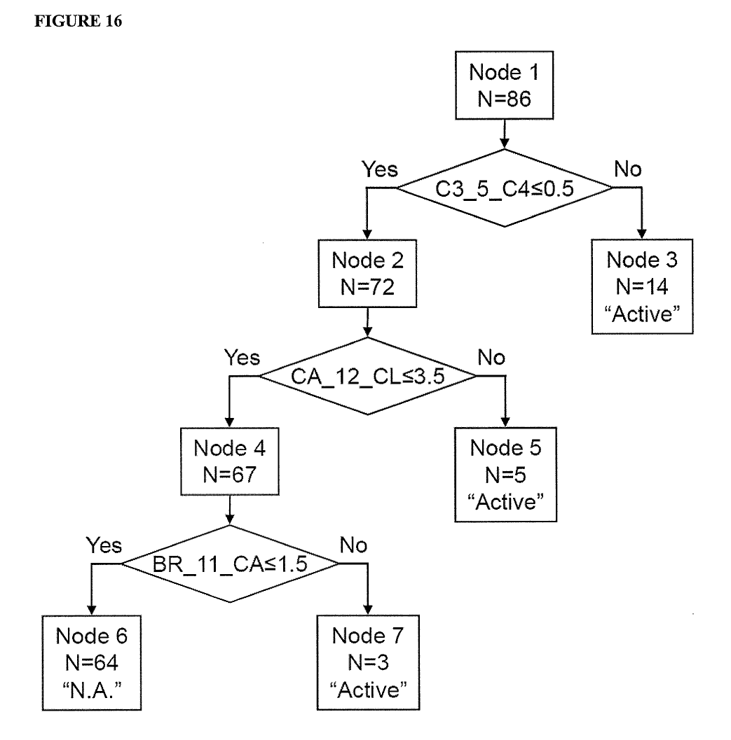

Based on the 13 descriptors selected in LDA above, the inventors obtained the optimal classification tree with univariate splits shown in FIG. 16. According to this tree, the prediction of Compounds 1-86 in Table 7 as “Active” or “NA” depends on three atom pairs: C3_{—}5_C4, CA_{—}12_CL, and BR_{—}11_CA (examples shown in FIG. 13). Taking into account that atom pair descriptors adopt integer values only, the conditions present in FIG. 16 can be interpreted as follows. If a compound has at least one C3_{—}5_C4 atom pair, then the compound is classified as “Active.”

Similarly, on the second and third splits, a compound is classified as “Active” if it has more than three CA_{—}12_CL atom pairs or more than one BR_{—}11_CA atom pair, respectively. An insufficient number of all the enumerated atom pairs leads to the left lowest terminal node where the compound is assigned as “NA.” In total, 84.9% of the compounds were classified correctly using only these three atom pairs (57 of 63 inactive and 16 of 23 active arylcarboxylic acid hydrazide derivatives were correctly classified) (Table 6). Classifications made by the tree for compounds 1-86 are shown in Table 7.

As indicated above, the BR_{—}11_CA atom pair represents in a “chemical” sense the simultaneous presence of a bromine atom and an aromatic ring on the opposite sides of a molecule. The descriptor C3_{—}5_C4 is characteristic of two types of compounds: one containing a methyl- or trifluoromethyl-substituted benzene ring attached to the furan moiety (see FIG. 13, Compounds 7 and 25), and the other containing an alkoxy group in the aromatic ring connected to the azomethine carbon of the linker (see FIG. 13, Compound 56). If activity is based on the presence of the CA_{—}12_CL atom pair, at least four of these atom pairs are necessary for classification as “Active.” This atom pair is present when aromatic fragments are located on both sides of a dumbbell shaped molecule, with one aromatic moiety containing two or more chlorine atoms in ortho and meta positions (four such atom pairs in Compound 24 are shown in FIG. 13).

Although the accuracy of classification by these simplified rules is slightly lower than that of the LDA or CTLCS approaches, it can be very useful for non-computational, logical prediction of the activity class for a given arylcarboxylic acid hydrazide derivative. Note that the classification tree model with univariate splits, like the LDA and CTLCS models, also includes “longer” atom pairs with 11 and 12 chemical bonds, which is in agreement with the proposed interaction of the entire non-metabolized molecule with a given biological target rather than smaller metabolites.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

All documents cited are incorporated herein by reference in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

### REFERENCES

Almasirad A, Hosseini R, Jalalizadeh H, Rahimi-Moghaddam Z, Abaeian N, Janafrooz M, Abbaspour M, Ziaee V, Dalvandi A and Shafiee A (2006) Synthesis and Analgesic Activity of 2-Phenoxybenzoic Acid and N-Phenylanthranilic Acid Hydrazides. Biol Pharm Bull 29:1180-1185.
Bae Y S, Lee H Y, Jo E J, Kim J I, Kang H K, Ye R D, Kwak J Y and Ryu S H (2004) Identification of Peptides That Antagonize Formyl Peptide Receptor-Like 1-Mediated Signaling. J Immunol 173:607-614.
Baguley B C (2001) Small-Molecule Cytokine Inducers Causing Tumor Necrosis. Curr Opin Investig Drugs 2:967-975.
Bertsch U, Winklhofer K F, Hirschberger T, Bieschke J, Weber P, Hartl F U, Tavan P, Tatzelt J, Kretzschmar H A and Giese A (2005) Systematic Identification of Antiprion Drugs by High-Throughput Screening Based on Scanning for Intensely Fluorescent Targets. J Virol 79:7785-7791.
Beutler B (1995) TNF, Immunity and Inflammatory Disease: Lessons of the Past Decade. J Invest Med 43:227-235.
Beutler B (2004) Innate Immunity: an Overview. Mol Immunol 40:845-859.
Boykin D W, Jr. and Varma R S (1970) Benzylidene Hydrazides As Potential Anticancer Agents. J Med Chem 13:583-584.
Burkhart C A, Berman J W, Swindell C S and Horwitz S B (1994) Relationship Between the Structure of Taxol and Other Taxanes on Induction of Tumor Necrosis Factor-Alpha Gene Expression and Cytotoxicity. Cancer Res 54:5779-5782.
Burli R W, Xu H, Zou X, Muller K, Golden J, Frohn M, Adlam M, Plant M H, Wong M, McElvain M, Regal K, Viswanadhan V N, Tagari P and Hungate R (2006) Potent HFPRL1 (ALXR) Agonists As Potential Anti-Inflammatory Agents. Bioorg Med Chem Lett 16:3713-3718.
Byrd-Leifer C A, Block E F, Takeda K, Akira S and Ding A (2001) The Role of MyD88 and TLR4 in the LPS-Mimetic Activity of Taxol. Eur J Immunol 31:2448-2457.
Edwards B S, Bologa C, Young S M, Balakin K V, Prossnitz E R, Savchuck N P, Sklar L A and Oprea T I (2005) Integration of Virtual Screening With High-Throughput Flow Cytometry to Identify Novel Small Molecule Formylpeptide Receptor Antagonists. Mol Pharm 68:1301-1310.
Elagoz A, Henderson D, Babu P S, Salter S, Grahames C, Bowers L, Roy M O, Laplante P, Grazzini E, Ahmad S and Lembo P M (2004) A Truncated Form of CKβ8-1 Is a Potent Agonist for Human Formyl Peptide-Receptor-Like 1 Receptor. Br J Pharmacol 141:37-46.
Engelhardt R, Mackensen A and Galanos C (1991) Phase I Trial of Intravenously Administered Endotoxin (Salmonella Abortus Equi) in Cancer Patients. Cancer Res 51:2524-2530.
Gao J L, Chen H, Filie J D, Kozak C A and Murphy P M (1998) Differential Expansion of the NFormylpeptide Receptor Gene Cluster in Human and Mouse. Genomics 51:270-276.
Gauss K A, Bunger P L, Larson T C, Young C J, Nelson-Overton L K, Siemsen D W and Quinn M T (2005) Identification of a Novel Tumor Necrosis Factor α-Responsive Region in the NCF2 Promoter. J Leukoc Biol 77:267-278.
Gauss K A, Nelson-Overton L K, Siemsen D W, Gao Y, DeLeo F R and Quinn M T (2007) Role of NF-Kappa B in Transcriptional Regulation of the Phagocyte NADPH Oxidase by Tumor Necrosis Factor-Alpha. J Leukoc Biol 82:729-741.
Gavins F N, Yona S, Kamal A M, Flower R J and Perretti M (2003) Leukocyte Antiadhesive Actions of Annexin 1: ALXR- and FPR-Related Anti-Inflammatory Mechanisms. Blood 101:4140-4147.
Gordon S (2007) The Macrophage: Past, Present and Future. Eur J Immunol 37 Suppl 1:S9-17. Guner O, Clement O and Kurogi Y (2004) Pharmacophore Modeling and Three Dimensional Database Searching for Drug Design Using Catalyst: Recent Advances. Curr Med Chem 11:2991-3005.
He R, Tan L, Browning D D, Wang J M and Ye R D (2000) The Synthetic Peptide Trp-Lys-Tyr-Met-Val-D-Met Is a Potent Chemotactic Agonist for Mouse Formyl Peptide Receptor. J Immunol 165:4598-4605.
Hermes-Lima M, Nagy E, Ponka P and Schulman H M (1998) The Iron Chelator Pyridoxal Isonicotinoyl Hydrazone (PIH) Protects Plasmid PUC-18 DNA Against *OH-Mediated Strand Breaks. Free Radic Biol Med 25:875-880.
Hoebe K, Janssen E and Beutler B (2004) The Interface Between Innate and Adaptive Immunity. Nat Immunol 5:971-974.
Hu J, Li G, Tong Y, Li Y, Zhou G, He X, Xie P, Wang J M and Sun Q (2005) Transduction of the Gene Coding for a Human G-Protein Coupled Receptor FPRL1 in Mouse Tumor Cells Increases Host Anti-Tumor Immunity. Int Immunopharmacol 5:971-980.
Hu J Y, Le Y, Gong W, Dunlop N M, Gao J L, Murphy P M and Wang J M (2001) Synthetic Peptide MMK-1 Is a Highly Specific Chemotactic Agonist for Leukocyte FPRL1. J Leukoc Biol 70:155-161.
Huang J, Hu J, Bian X, Chen K, Gong W, Dunlop N M, Howard O M and Wang J M (2007) Transactivation of the Epidermal Growth Factor Receptor by Formylpeptide Receptor Exacerbates the Malignant Behavior of Human Glioblastoma Cells. Cancer Res 67:5906-5913.
Ikeda Y, Murakami A, Fujimura Y, Tachibana H, Yamada K, Masuda D, Hirano K, Yamashita S and Ohigashi H (2007) Aggregated Ursolic Acid, a Natural Triterpenoid, Induces IL-1beta Release From Murine Peritoneal Macrophages: Role of CD36. J Immunol 178:4854-4864.
Inoue M, Ueda M, Ogihara Y and Saracoglu I (1998) Induction of Cytokines by a Phenylpropanoid Glycoside Acteoside. Biol Pharm Bull 21:1394-1395.
Jaeckle K A, Mittelman A and Hill F H (1990) Phase II Trial of Serratia Marcescens Extract in Recurrent Malignant Astrocytoma. J Clin Oncol 8:1408-1418.
Joseph W R, Cao Z, Mountjoy K G, Marshall E S, Baguley B C and Ching L M (1999) Stimulation of Tumors to Synthesize Tumor Necrosis Factor-α in Situ Using 5,6-Dimethylxanthenone-4-Acetic Acid: a Novel Approach to Cancer Therapy. Cancer Res 59:633-638.
Katakami Y, Nakao Y, Koizumi T, Katakami N, Ogawa R and Fujita T (1988) Regulation of Tumour Necrosis Factor Production by Mouse Peritoneal Macrophages: the Role of Cellular Cyclic AMP. Immunology 64:719-724.
Kim Y, Lee B D, Kim O, Bae Y S, Lee T, Suh P G and Ryu S H (2006) Pituitary Adenylate Cyclase-Activating Polypeptide 27 Is a Functional Ligand for Formyl Peptide Receptor-Like 1. J Immunol 176:2969-2975.
Kirikae T, Ojima I, Fuero-Oderda C, Lin S, Kirikae F, Hashimoto M and Nakanoc M (2000) Structural Significance of the Acyl Group at the C-10 Position and the A Ring of the Taxane Core of Paclitaxel for Inducing Nitric Oxide and Tumor Necrosis Factor Production by Murine Macrophages. FEBS Lett 478:221-226.
Le Y, Hu J, Gong W, Shen W, Li B, Dunlop N M, Halverson D O, Blair D G and Wang J M (2000) Expression of Functional Formyl Peptide Receptors by Human Astrocytoma Cell Lines. J Neuroimmunol 111: 102-108.
Lejeune F J, Lienard D, Matter M and Ruegg C (2006) Efficiency of Recombinant Human TNF in Human Cancer Therapy. Cancer Immun 6:6.
Mayne M, Holden C P, Nath A and Geiger J D (2000) Release of Calcium From Inositol 1,4,5-Trisphosphate Receptor-Regulated Stores by HIV-1 Tat Regulates TNF-α Production in Human Macrophages. J Immunol 164:6538-6542.
Migeotte I, Communi D and Parmentier M (2006) Formyl Peptide Receptors: a Promiscuous Subfamily of G Protein-Coupled Receptors Controlling Immune Responses. Cytokine Growth Factor Rev 17:501-519.
Nanamori M, Cheng X, Mei J, Sang H, Xuan Y, Zhou C, Wang M W and Ye R D (2004) A Novel Nonpeptide Ligand for Formyl Peptide Receptor-Like 1. Mol Pharm 66:1213-1222.
Nemunaitis J, Vukelja S J, Richards D, Cunningham C, Senzer N, Nugent J, Duncan H, Jones B, Haltom E and Uprichard M J (2006) Phase I Trial of PT-100 (PT-100), a Cytokine-Inducing Small Molecule, Following Chemotherapy for Solid Tumor Malignancy. Cancer Invest 24:553-561.
Nicklaus M C, Wang S M, Driscoll J S and Milne G W A (1995) Conformational-Changes of Small Molecules Binding to Proteins. Bioorg Med Chem 3:411-428.
Old L J (1985) Tumor Necrosis Factor (TNF). Science 230:630-632.
Perretti M, Chiang N, La M, Fierro I M, Marullo S, Getting S J, Solito E and Serhan C N (2002) Endogenous Lipid- and Peptide-Derived Anti-Inflammatory Pathways Generated With Glucocorticoid and Aspirin Treatment Activate the Lipoxin A4 Receptor. Nat Med 8:1296-1302.
Pollaud-Chemon C, Vandaele J, Quartulli F, Seguelas M H, Decerprit J and Pipy B (1998) Involvement of Calcium and Arachidonate Metabolism in Acetylated-Low-Density-Lipoprotein-Stimulated Tumor-Necrosis-Factor-α Production by Rat Peritoneal Macrophages. Eur J Biochem 253:345-353.
Rabiet M J, Huet E and Boulay F (2007) The N-Formyl Peptide Receptors and the Anaphylatoxin C5a Receptors: An Overview. Biochimie 89:1089-1106.
Reed J C (2006) Drug Insight: Cancer Therapy Strategies Based on Restoration of Endogenous Cell Death Mechanisms. Nat Clin Pract Oncol 3:388-398.
Rescher U, Danielczyk A, Markoff A and Gerke V (2002) Functional Activation of the Formyl Peptide Receptor by a New Endogenous Ligand in Human Lung A549 Cells. J Immunol 169:1500-1504.
Schepetkin I A, Faulkner C L, Nelson-Overton L K, Wiley J A and Quinn M T (2005) Macrophage Immunomodulatory Activity of Polysaccharides Isolated From Juniperus Scopolorum. Int Immunopharmacol 5:1783-1799.
Schepetkin I A, Kirpotina L N, Khlebnikov A I and Quinn M T (2007) High-Throughput Screening for Small-Molecule Activators of Neutrophils: Identification of Novel N-Formyl Peptide Receptor Agonists. Mol Pharm 71:1061-1074.
Schiller M, Metze D, Luger T A, Grabbe S and Gunzer M (2006) Immune Response Modifiers—Mode of Action. Exp Dermatol 15:331-341.
Schön M and Schön M P (2007a) The Antitumoral Mode of Action of Imiquimod and Other Imidazoquinolines. Curr Med Chem 14:681-687.
Schön M P and Schön M (2007b) Imiquimod: Mode of Action. Br J Dermatol 157 Suppl 2:8-13. Serhan CN (2007) Resolution Phase of Inflammation: Novel Endogenous Anti-Inflammatory and Proresolving Lipid Mediators and Pathways. Annu Rev Immunol 25:101-137.
Simunek T, Boer C, Bouwman R A, Vlasblom R, Versteilen A M, Sterba M, Gersl V, Hrdina R, Ponka P, de Lange J J, Paulus W J and Musters R J (2005) SIH—a Novel Lipophilic Iron Chelator—Protects H9c2 Cardiomyoblasts From Oxidative Stress-Induced Mitochondrial Injury and Cell Death. J Mol Cell Cardiol 39:345-354.
Tosi M F (2005) Innate Immune Responses to Infection. J Allerg Clin Immunol 116:241-249.
Vulcano M, Rosa M F A, Minnucci F S, Cherñavsky A C and Isturiz M A (1998) N-Formyl-Methionyl-Leucyl-Phenylalanine (FMLP) Inhibits Tumour Necrosis Factor-Alpha (TNF-α) Production on Lipopolysaccharide (LPS)-Stimulated Human Neutrophils. Clin Exp Immunol 113:39-47.
Wagner T L, Horton V L, Carlson G L, Myhre P E, Gibson S J, Imbertson L M and Tomai M A (1997) Induction of Cytokines in Cynomolgus Monkeys by the Immune Response Modifiers, Imiquimod, S-27609 and S-28463. Cytokine 9:837-845.
Wang J, Kobayashi M, Han M, Choi S, Takano M, Hashino S, Tanaka J, Kondoh T, Kawamura K and Hosokawa M (2002) MyD88 Is Involved in the Signalling Pathway for Taxol-Induced Apoptosis and TNF-α Expression in Human Myelomonocytic Cells. Br J Haematol 118:638-645.
Watanabe N, Suzuki J and Kobayashi Y (1996) Role of Calcium in Tumor Necrosis Factor-α Production by Activated Macrophages. J Biochem 120:1190-1195.
Yang D, Chen Q, Schmidt A P, Anderson G M, Wang J M, Wooters J, Oppenheim J J and Chertov O (2000) LL-37, the Neutrophil Granule- and Epithelial Cell-Derived Cathelicidin, Utilizes Formyl Peptide Receptor-Like 1 (FPRL1) As a Receptor to Chemoattract Human Peripheral Blood Neutrophils, Monocytes, and T Cells. J Exp Med 192:1069-1074.
Zhang H Z, Drewe J, Tseng B, Kasibhatla S and Cai S X (2004) Discovery and SAR of Indole-2-Carboxylic Acid Benzylidene-Hydrazides As a New Series of Potent Apoptosis Inducers Using a Cell-Based HTS Assay. Bioorg Med Chem 12:3649-3655.
Zhang X and Morrison D C (1993) Pertussis Toxin-Sensitive Factor Differentially Regulates Lipopolysaccharide-Induced Tumor Necrosis Factor-α and Nitric Oxide Production in Mouse Peritoneal Macrophages. J Immunol 150:1011-1018.
Zhelev D V and Alteraifi A (2002) Signaling in the Motility Responses of the Human Neutrophil. Ann Biomed Eng 30:356-370.
Zhou C, Zhang S, Nanamori M, Zhang Y, Liu Q, Li N, Sun M, Tian J, Ye P P, Cheng N, Ye R D and Wang M W (2007) Pharmacological Characterization of a Novel Nonpeptide Antagonist for Formyl Peptide Receptor-Like 1. Mol Pharmacol 72:976-983.
Zhou Y, Bian X, Le Y, Gong W, Hu J, Zhang X, Wang L, Iribarren P, Salcedo R, Howard O M, Farrar W and Wang J M (2005) Formylpeptide Receptor FPR and the Rapid Growth of Malignant Human Gliomas. J Natl Cancer Inst 97:823-835.
Wagner, G.; Laufer, S. Med Res. Rev. 2006, 26, 1.
Baguley, B. C. Curr. Opin. Investig. Drugs 2001, 2, 967.
Schepetkin, I. A.; Kirpotina, L. N.; Tian, J.; Khlebnikov, A. I.; Ye, R. D.; Quinn, M. T. Mol. Pharm. 2008, In press.
Andricopulo, A. D.; Montanari, C. A. Mini. Rev. Med. Chem. 2005, 5, 585.
Kirikae, T.; Ojima, I.; Kirikae, F.; Ma, Z.; Kuduk, S. D.; Slater, J. C.; Takeuchi, C. S.; Bounaud, P. Y.; Nakano, M. Biochem. Biophys. Res. Commun. 1996, 227, 227.
Ojima, I.; Fumero-Oderda, C. L.; Kuduk, S. D.; Ma, Z.; Kirikae, F.; Kirikae, T. Bioorg. Med. Chem. 2003, 11, 2867.
Buttingsrud, B.; Ryeng, E.; King, R. D.; Alsberg, B. K. J. Comput. Aided Mol. Des 2006, 20, 361.
Khlebnikov, A. I.; Schepetkin, I. A.; Domina, N. G.; Kirpotina, L. N.; Quinn, M. T. Bioorg. Med. Chem. 2007, 15, 1749.
Khlebnikov, A. I.; Schepetkin, I. A.; Quinn, M. T. Bioorg. Med. Chem. 2008, 16, 2791.
Carhart, R. E.; Smith, D. H.; Venkataraghavan, R. J. Chem. Inf. Comput. Sci. 1985, 25, 64.
Lindman, H. R. Analysis of Variance in Complex Experimental Designs; W. H. Freeman & Co.: San Francisco, 1974.
Loh, W. Y.; Shih, Y. S. Statistica Sinica 1997, 7, 815.
Breiman, L.; Friedman, J. H.; Olshen, R. A.; Stone, C. J. Classification and Regression Trees; Wadsworth & Brooks/Cole Advanced Books & Software: Monterey, 1984.
Chapoval et al., 1998, J. Immunol. 161:6977-6984
Gollub et al., 1998, J. Clin. Invest. 102:561-575
Kikuchi et al., 1999, Int. J. Cancer 80:425-430
Pan et al., 1995, Int. J. Cancer 80:425-430
Saffran et al., 1998, Cancer Gene Ther. 5:321-330
Zimmermann et al., 1999, Eur. J. Immunol. 29:284-290.

1. A compound of structural formula (I): 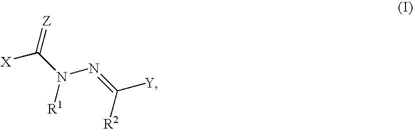
or a salt, solvate, ester and/or prodrug thereof,
wherein,
X and Y are each independently aryl, substituted aryl, heteroaryl, or substituted heteroaryl;
Z is oxygen or sulfur;
R¹ is hydrogen or C1 to C6 alkyl; and
R² is hydrogen, C1 to C6 alkyl, or R² and Y, taken together with the carbon atom to which they are bonded, form a substituted heterocyclic ring.

2. The compound of claim 1, wherein Z is oxygen.

3. The compound of claim 1, wherein R¹ is hydrogen.

4. The compound of claim 1, wherein R² is hydrogen.

5. The compound of claim 1, wherein Z is oxygen; R¹ is hydrogen; and R² is hydrogen.

6. The compound of claim 1, wherein Z is oxygen; R¹ is hydrogen; and R² and Y, taken together with the carbon atom to which they are bonded, form a substituted heterocyclic ring.

7. The compound of claim 1, wherein aryl is phenyl or naphthyl.

8. The compound of claim 1, wherein substituted aryl comprises one or more substituents selected from the group consisting of alkyl, substituted alkyl, aryl, substituted aryl, heteroaryl, substituted heteroaryl, hydroxy, halo, nitro, cyano, amino, alkylamino, —OR³, and a combination thereof; and optionally two of the substituents, taken together with the carbon atoms to which they are bonded, form a heterocyclic ring;
wherein R³ is alkyl, arylalkyl, heteroarylalkyl, (aryl)-C(O)—, (substituted aryl)-C(O)—, (heteroaryl)-C(O)—, or (substituted heteroaryl)-C(O)—.

9. The compound of claim 8, wherein two of the substituents, taken together with the carbon atoms to which they are bonded, form a dioxole or dioxine.

10. The compound of claim 1, wherein heteroaryl comprises a 5- or 6-membered aromatic ring having 1 to 3 heteroatoms selected from the group consisting of nitrogen, oxygen, sulfur, and a combination thereof.

11. The compound of claim 10, wherein heteroaryl is selected from the group consisting of furan, pyrrole, thiophene, imidazole, oxazole, thiazole, pyridine, pyrazine, and pyrimidine.

12. The compound of claim 1, wherein substituted heteroaryl comprises one or more substituents selected from the group consisting of alkyl, substituted alkyl, aryl, substituted aryl, heteroaryl, substituted heteroaryl, hydroxy, halo, nitro, cyano, amino, alkylamino, —OR³, and a combination thereof; and optionally two of the substituents, taken together with the carbon atoms to which they are bonded, form a heterocyclic ring;
wherein R³ is alkyl, arylalkyl, heteroarylalkyl, (aryl)-C(O)—, (substituted aryl)-C(O)—, (heteroaryl)-C(O)—, or (substituted heteroaryl)-C(O)—.

13. The compound of claim 1, wherein
X is aryl, substituted aryl, heteroaryl, or substituted heteroaryl; wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof;
Y is heteroaryl or substituted heteroaryl; wherein substituted heteroaryl comprises one or more substituents selected from the group consisting of alkyl, substituted alkyl, aryl, substituted aryl, heteroaryl, substituted heteroaryl, alkoxy, hydroxy, halo, nitro, cyano, amino, alkylamino, and a combination thereof;
Z is oxygen; and
R¹ and R² are both hydrogen.

14. The compound of claim 1, wherein
X is substituted aryl which comprises two or more substituents, wherein two of the substituents, taken together with the carbon atoms to which they are bonded, form a heterocyclic ring;
Y is heteroaryl or substituted heteroaryl; wherein substituted heteroaryl comprises one or more substituents selected from the group consisting of alkyl, substituted alkyl, aryl, substituted aryl, heteroaryl, substituted heteroaryl, alkoxy, hydroxy, halo, nitro, cyano, amino, alkylamino, and a combination thereof;
Z is oxygen; and
R¹ and R² are both hydrogen.

15. The compound of claim 1, wherein the compound has structural formula (II): 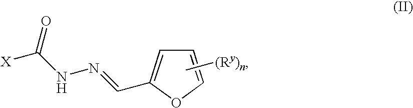
wherein,
X is aryl, substituted aryl, heteroaryl, or substituted heteroaryl;
each R^{y} is independently alkyl, substituted alkyl, aryl, substituted aryl, heteroaryl, substituted heteroaryl, alkoxy, hydroxy, halo, nitro, cyano, amino, or alkylamino; and
n is 0, 1, 2, or 3.

16. The compound of claim 15, wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof; and optionally two of the substituents, taken together with the carbon atoms to which they are bonded, form a heterocyclic ring;

17. The compound of claim 1, wherein
X is aryl, substituted aryl, heteroaryl, or substituted heteroaryl; wherein substituted aryl and substituted heteroaryl each independently comprises one or more substituents selected from the group consisting of alkyl, substituted alkyl, hydroxy, alkoxy, halo, nitro, cyano, amino, alkylamino, and a combination thereof;
Y is aryl or substituted aryl; wherein substituted aryl comprises one or more substituents selected from the group consisting of alkyl, substituted alkyl, hydroxy, halo, nitro, cyano, amino, alkylamino, —OR³, and a combination thereof; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—;
Z is oxygen; and
R¹ and R² are both hydrogen.

18. The compound of claim 1, wherein
X is substituted aryl which comprises two or more substituents, wherein two of the substituents, taken together with the carbon atoms to which they are bonded, form a heterocyclic ring;
Y is aryl or substituted aryl; wherein substituted aryl comprises one or more substituents selected from the group consisting of alkyl, substituted alkyl, hydroxy, halo, nitro, cyano, amino, alkylamino, —OR³, and a combination thereof; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—;
Z is oxygen; and
R¹ and R² are both hydrogen.

19. The compound of claim 1, wherein the compound has structural formula (III): 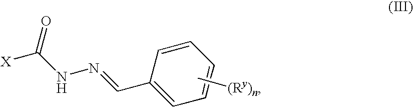
wherein,
X is aryl, substituted aryl, heteroaryl, or substituted heteroaryl;
each R^{y} is independently alkyl, substituted alkyl, hydroxy, halo, nitro, cyano, amino, alkylamino, or —OR³; wherein R³ is alkyl, arylalkyl, heteroarylalkyl, (aryl)-C(O)—, (substituted aryl)-C(O)—, (heteroaryl)-C(O)—, or (substituted heteroaryl)-C(O)—; and
n is 0, 1, 2, or 3.

20. The compound of claim 1, wherein
X is substituted aryl, which comprises two or more substituents wherein two of the substituents, taken together with the carbon atoms to which they are bonded, form a heterocyclic ring;
Z is oxygen;
R¹ is hydrogen; and
R² and Y, taken together with the carbon atom to which they are bonded, form a substituted heterocyclic ring.

21. The compound of claim 1, which is selected from the group consisting of

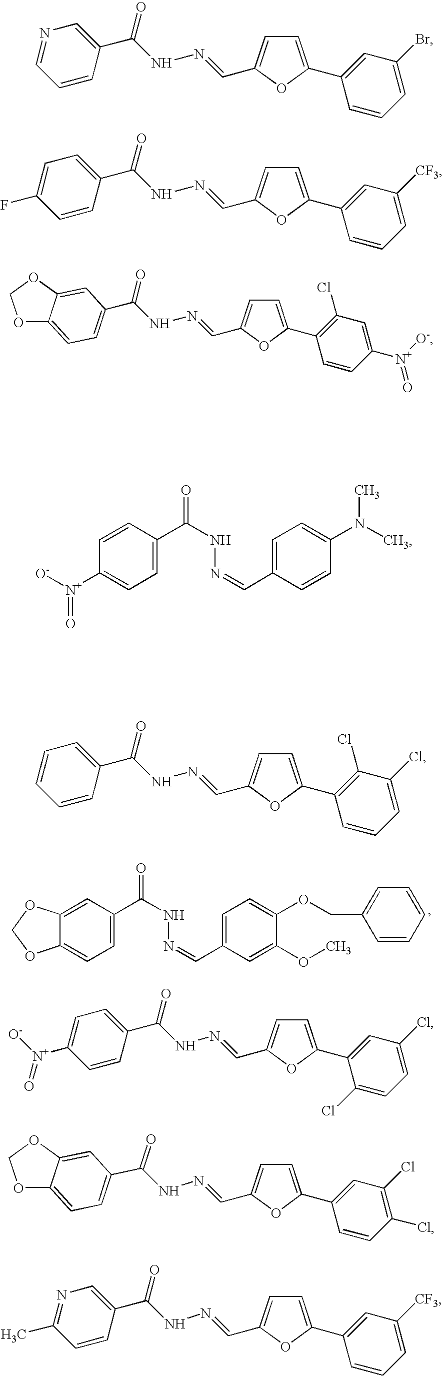

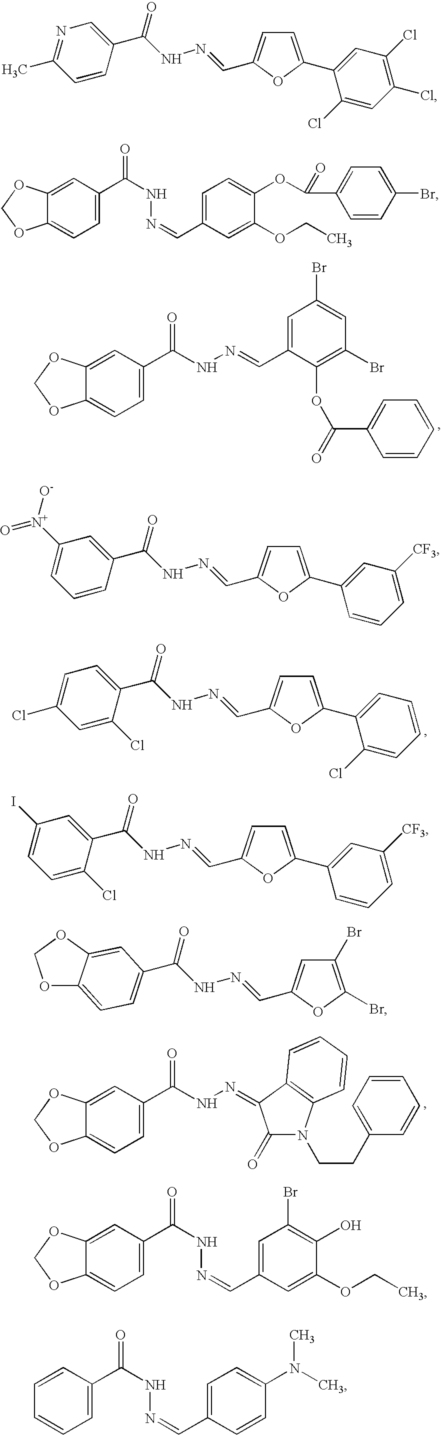

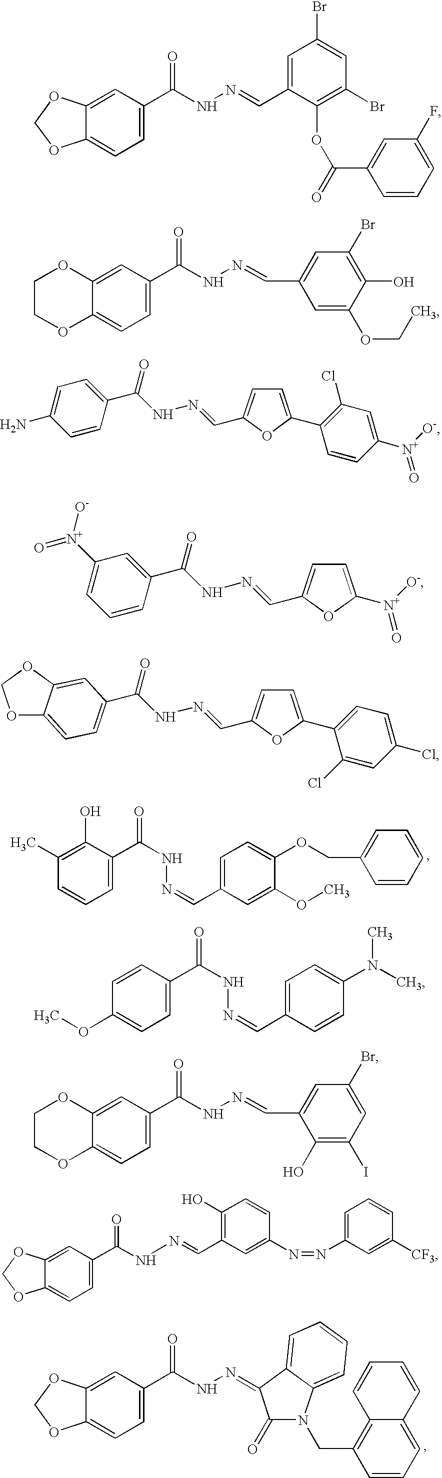

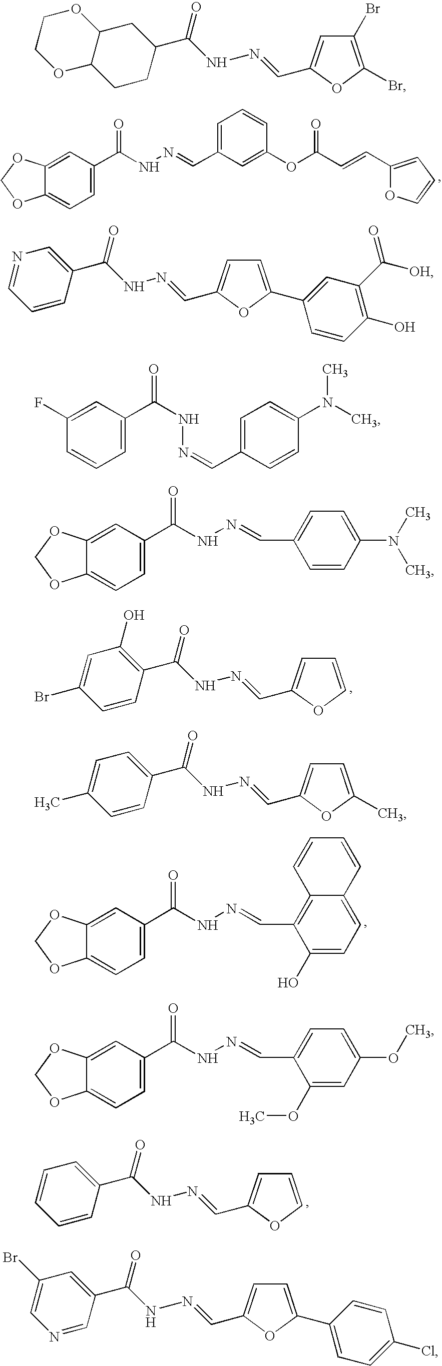

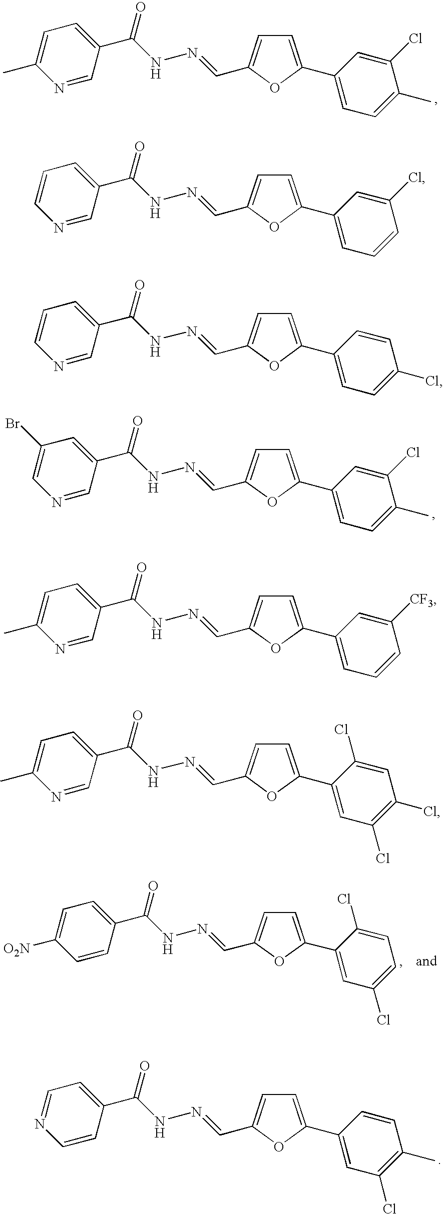

22. A pharmaceutical composition comprising
a therapeutically effective amount of the compound of claim 1, or a salt, solvate, ester, and/or prodrug thereof; and
a pharmaceutically acceptable carrier.

23. A method of activating N-formyl peptide receptors (FPR) in a cell comprising contacting the cell with an effective amount of the compound of claim 1, or a salt, solvate, ester, and/or prodrug thereof.

24. A method of stimulating production of tumor necrosis factor α (TNF-α) in a cell comprising contacting the cell with an effective amount of the compound of claim 1, or a salt, solvate, ester, and/or prodrug thereof.

25. A method of inducing apoptosis in a tumor-associated cell comprising contacting the tumor-associated cell with an effective amount of the compound of claim 1, or a salt, solvate, ester, and/or prodrug thereof.

26. A method of treating a disease, condition, or symptom associated with TNF-α for a patient in need thereof comprising administering to the patient a therapeutically effective amount of the compound of claim 1, or a salt, solvate, ester, and/or prodrug thereof.

27. The method of claim 26, wherein the disease, condition, or symptom is a neoplastic disease.
